(19) United States
(12) Patent Application Publication
(10) Pub. No.: US 2023/0165891 A1
(43) Pub. Date: Jun. 1, 2023

(54) VIRUCIDAL COMPOSITIONS AND USE THEREOF

(71) Applicant: ECOLE POLYTECHNIQUE FEDERALE DE LAUSANNE (EPFL), Lausanne (CH)

(72) Inventors: Matteo GASBARRI, Lausanne (CH); Francesco STELLACCI, Morges (CH); Ehsan MOHAMMADIFAR, Berlin (DE); Rainer HAAG, Berlin (DE)

(21) Appl. No.: 18/160,650

(22) Filed: Jan. 27, 2023

Related U.S. Application Data
(63) Continuation-in-part of application No. PCT/EP2021/071188, filed on Jul. 28, 2021.
Application No. PCT/EP2021/071188 is a continuation-in-part of application No. PCT/EP2021/070990, filed on Jul. 27, 2021.

(30) Foreign Application Priority Data
Jul. 30, 2020 (EP) 20188654.6

(51) Int. Cl. A61K 31/765; A61P 31/22; A01N 31/02; A01P 1/00; A61P 31/14; A61K 31/795; A01N 41/04; C08G 65/48

(52) U.S. Cl. CPC A61K 31/765; A61P 31/22; A01N 31/02; A01P 1/00; A61P 31/14; A61K 31/795; A01N 41/04; C08G 65/48

(57) ABSTRACT

The disclosure relates to dendritic polyglycerols (dPG) compounds with carboxyalkyl, sulfyl or sulfonyl functional groups that irreversibly inhibit viral infection (virucidal effect) through multivalent interaction in nanomolar concentration range. While the compounds of the disclosure show virus inhibition in the nanomolar range they show no in-vitro toxicity in the same range of concentration.

This application is a continuation-in-part of International Application No. PCT/EP2021/071188, filed Jul. 28, 2021, and International Application No. PCT/EP2021/070990, filed Jul. 27, 2021, both of which claim the benefit of priority of European Application No. 20188654.6, filed Jul. 30, 2020, the disclosures of each are hereby incorporated by reference as if written herein in their entireties.

### FIELD OF THE DISCLOSURE

This disclosure relates to dendritic polyglycerol (dPG) compounds having alkyl-carboxylate, -sulfate or -sulfonate functional groups that irreversibly inhibit viruses and are useful in the treatment of viral infections such as COVID-19.

### BACKGROUND OF THE DISCLOSURE

The recent emergence of SARS-CoV-2 resulted in a global pandemic (COVID-19), threatening the health of the world's population and causing dramatic socio-economic damage. It is known that new viruses can emerge or re-emerge every 3-4 years, as previously shown by H1N1, Ebola, H5N1, Zika, etc., all episodes that revealed how our society is unprepared to respond to novel viruses. Indeed, even the percentages of people infected by known viruses such as HSV, HIV, and influenza evidence the urgency of developing novel strategies in fighting viral diseases.

At present, there are two primary weapons against viruses: vaccines and antivirals. Vaccines are preventive drugs composed of modified or attenuated pathogens that are meant to stimulate an immunological bio-response prior to exposure to a live virus. At the moment, vaccines represent the most effective approach to preventing viral infections. However, the durability of protection following vaccination is not 100%. Vaccines are not always available, particularly in underdeveloped countries, and existing vaccines are highly unlikely to be effective against a virus that has not yet emerged. Thus, there remains a large unmet medical need for therapeutic interventions that can help at-risk and infected individuals. Antivirals are drugs designed to fight against viruses and viral infections directly.

The life cycle of a virus is composed of multiple steps: 1) attachment, 2) entry, 3) uncoating, 4) biosynthesis, and 5) assembly and release. The typical mechanism of action of existing antivirals involves inhibiting a step of the viral life cycle, thereby stopping replication. Most antivirals target one or more of steps 2-5, requiring each antiviral to be specific for the manner in which such step is carried out by a particular virus. Given the error-prone nature of viral replication, viruses are often known to mutate and develop resistance to antivirals.

The first step of the viral life cycle is attachment. In this step the virus recognizes a host cell using receptors on viral attachment ligands (VALs) that recognize and bind to specific proteins present on host cell membranes. It is known that the VALs of a significant percentage of all viruses target either heparane sulfate proteoglycans (HSPG) or sialic acid (SA) terminal moieties of proteins present on cell membranes. This facilitates a different approach to designing antivirals by mimicking HSPG or SA with a molecule (ranging from polymers to dendrimers, oligomers, nanoparticles, liposomes, monoclonal antibodies, and small molecules) that will bind to a virus and block viral entry. Many of these compounds have shown broad-spectrum activity and limited toxicity, yet none has been translated into a successful drug. The main limitation of such binding inhibitors lies in their mechanism itself. Binding is a reversible event, particularly when the environment (e.g., the bloodstream) surrounding a compound that is bound to a virus causes dissociation of the virus-compound complex, separating the virus from the compound that prevented binding and leaving the virus free to bind again. Unfortunately, dilution is a common event, especially in vivo. Such temporary blocking of viral attachment and/or replication is referred to as virustatic.

The irreversible inhibition of the infectivity of a virus following interaction with an antiviral compound or composition is referred to as virucidal. Many known compounds, ranging from strong surfactants to alcohol, can irreversibly inhibit the infectivity of viruses. Most of these compounds, however, have not translated into acceptable drugs due to issues such as toxicity. Viruses are made of components similar to those of the host, so a drug that damages or interferes with such common components in a virus or an infected cell will also damage (i.e., be toxic to) the host. Only a few compounds have demonstrated virucidal properties together with low toxicity, such as certain reported peptides, but these have been virus-specific, not broad spectrum.

Therefore, there is still a need for virucidal agents that have low toxicity, excellent virucidal potency and broad-spectrum action.

### SUMMARY

In accordance with the objects outlined above, the present disclosure provides compositions and methods that can be used to treat viral diseases, e.g., COVID-19.

In one aspect, the disclosure provides a compound, pharmaceutically acceptable salt or pharmaceutically acceptable ester of Formula I:

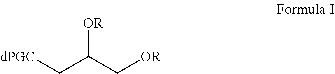

dPGC is dendritic polyglycerol core in which OR represents the free OH groups within and at the peripheryu of the core, having an average molecular weight from 5 to 100 kDa as measured by GPC;

R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH, —SO₃⁻, —(CH₂)_{z}—S—(CH₂)_{y}—CH₃, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃⁻, —(C₅ to C₃₀ alkenyl)-SO₃⁻, -(optionally substituted C₅ to C₃₀ alkyl)-O—SO₃⁻, —(C₅ to C₃₀ alkenyl)-OSO₃⁻, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃⁻;

y is an integer from about 4 to about 30;

z is an integer from 1 or 2 to about 20;

y+z is an integer from about 5 or 6 to about 30; and

having a DF of at least about 30%,

Alternatively, in Formula I,

dPGC is dendritic polyglycerol core having an average molecular weight from 5 to 100 kDa as measured by GPC;

R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH;

y is an integer from about 4 to about 30;

z is an integer from 1 to about 20;

y+z is an integer from about 5 to about 30; and

having a DF of at least about 30% as measured by ¹HNMR, where R contributing to the DF has a —COOH.

Alternatively, in Formula I,

dPGC is dendritic polyglycerol core having an average molecular weight from 5 to 100 kDa as measured by GPC;

R can be the same or different and is selected from the group comprising: —H, —SO₃⁻, —(CH₂)_{z}—S—(CH₂)_{y}—CH₃, -optionally substituted C₅ to C₃₀ alkyl, —C₃ to C₃₀ alkenyl, C₅ to C₃₀ ω-hydroxyalkyl, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃—, —(C₅ to C₃₀ alkenyl)-SO₃⁻, -(optionally substituted C₅ to C₃₀ alkyl)-OSO₃, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃;

y is an integer from about 4 to about 30;

z is an integer from about 2 to about 20;

y+z is an integer from about 6 to about 30; and

having a DF of at least about 30% as measured by 1 HNMR, where R contributing to the DF has an —SO₃ or an —O—SO₃⁻.

In Formula I, “OR” represents the free OH groups within and at the periphery of the core.

The compounds of Formula I are useful as active agents in practice of the methods of treatment and in manufacture of the pharmaceutical formulations of the disclosure, and as intermediates in the synthesis of such active agents.

Another aspect of the present disclosure provides a pharmaceutical composition comprising an effective amount of the one or more compounds of the disclosure and at least one pharmaceutically acceptable excipient, carrier and/or diluent.

Another aspect of the present disclosure provides the compounds of the disclosure for use in treating and/or preventing viral infections and/or diseases associated with viruses, such as SARS-CoV-2.

Another aspect of the present disclosure provides a virucidal composition comprising an effective amount of one or more compounds of the disclosure and at least one suitable carrier or aerosol carrier.

Another aspect of the present disclosure provides a device comprising the virucidal composition of the disclosure or one or more compounds of the disclosure and means for applying and/or dispensing thereof.

Another aspect of the present disclosure provides a method of disinfection and/or sterilization of non-living surfaces using one or more compounds of the disclosure or the virucidal composition of the disclosure.

Another aspect of the present disclosure provides a use of one or more compounds of the disclosure or the virucidal composition of the disclosure for sterilization and/or for disinfection of human or animal skin and/or hair.

Another aspect of the present disclosure provides a use of one or more compounds of the disclosure or the virucidal composition of the disclosure for manufacturing virucidal surfaces.

Another aspect of the present disclosure provides a device comprising a surface coated with one or more compounds of the disclosure or with the virucidal composition of the disclosure.

### BRIEF DESCRIPTION OF THE FIGURES

FIG. 1 shows a dendritic polyglycerol core and a route for its synthesis by anionic ring opening of glycidol.

FIG. 2 illustrates another dendritic polyglycerol core.

FIG. 3 shows the hydrodynamic diameter measured by DLS (dynamic light scattering) of the non-functionalized core dPG (C1) in phosphate buffer at concentration 1 mg/mL. The results are for three measurements.

FIG. 4 shows gel permeation chromatography (GPC) diagram of the non-functionalized core dPG (C1). (Mn:7.2 kDa, Mw: 10 kDa, PDI: 1.4)

FIG. 5 shows the ¹HNMR of dPG_{10 kDa}-C₃SC₁₀—COO⁻Na⁺_{100%} in D₂O.

FIG. 6 shows the hydrodynamic diameter measured by DLS (dynamic light scattering) of dPG_{10 kDa}-C₃SC₁₀—COO⁻Na⁺_{95%} in water.

FIG. 7 shows the ¹HNMR and DLS characterizing data for dPG_{5 kDa}-C₃SC₁₀—COO⁻Na⁺_{100%} (“EM124”).

FIG. 8 shows the ¹HNMR and DLS characterizing data for dPG_{25 kDa}-C₃SC₁₀—COO⁻Na⁺_{100%} (“EM126”).

FIG. 9 shows the ¹HNMR and DLS characterizing data for dPG_{100 kDa}-C₃SC₁₀—COO⁻Na⁺_{100%} (“EM131”).

FIG. 10 shows the ¹HNMR and DLS characterizing data for dPG_{5 kDa}-C₃SC₁₁—COO⁻Na⁺_{100%} (“EM125”).

FIG. 11 shows the SARS-CoV-2 antiviral activity and virucidal activity of dPG_{10 kDa}-C₃SC₁₀—COO⁻Na⁺_{100%}, along with the cytotoxicity data for the test compound.

FIG. 12 shows the SARS-CoV-2 antiviral and virucidal activity of dPG_{5 kDa}-C₃SC₁₀—COO⁻Na⁺_{100%} (“EM124”).

FIG. 13 shows the SARS-CoV-2 antiviral and virucidal activity of dPG_{5 kDa}-C₃SC₁₁—COO⁻Na⁺_{100%} (“EM125”).

FIG. 14 shows the SARS-CoV-2 antiviral and virucidal activity of dPG_{25 kDa}-C₃SC₁₀—COO⁻Na⁺_{100%} (“EM126”).

FIG. 15 shows the SARS-CoV-2 antiviral and virucidal activity of dPG_{100 kDa}-C₃SC₁₀—COO⁻Na⁺_{100%} (“EM131”).

FIG. 16 shows the HSV-2 antiviral activity and virucidal activity of dPG_{10 kDa}-C₃SC₁₀—COO⁻Na⁺_{95%}.

FIG. 17 shows the the SARS-CoV-2 in vivo antiviral activity of dPG_{10 kDa}-C₃SC₁₀—COO⁻Na⁺_{100%} as tested in the Syrian hamster model. The figure shows the change in percentage of body weight relative to each half day (each point on x-axys is 12 h). End-point day 14.

FIG. 18 shows functionalization of dendritic polyglycerols by allyl bromide. The dendritic polyglycerol core is shown as a solid sphere.

FIG. 19 shows the ¹HNMR spectra of dPG_{10 kDa}-allyl.

FIG. 20 shows a synthetic route for the synthesis of 11-mercapto-1-undecanesulfonate (MUS).

FIG. 21 shows ¹HNMR of 11-mercapto-1-undecanesulfonate (MUS).

FIG. 22 shows synthetic route for the synthesis dPG-MUS.

FIG. 23 shows ¹HNMR spectra of MUS (top), dPG-allyl (middle) and dPG-MUS (bottom).

FIG. 24 shows two-step approach for the synthesis of the dPG-C11-sulfate_{50%} (RX, R21).

FIG. 25 shows ¹HNMR spectrum of dPG-C11-sulfate (RX).

FIG. 26 shows two-step approach for the synthesis of the dPG-C11-sulfate_{50%} (R19B).

FIG. 27 shows ¹HNMR spectrum of dPG-C11-sulfate (R19B).

FIG. 28 shows one-pot approach for the synthesis of the a) dPG-C₄-sulfonate (RP3B) and b) dPG-C₃-sulfate (RN4) along with ¹HNMR characterizing data.

FIG. 29 shows ¹HNMR of dPG-C₄-sulfonate (top) and dPG-C₃-sulfate (bottom).

FIG. 30 shows on the top, dose-response curve of dendritic polymer 2 (solid circles), R17 (solid triangles), R18 (solid squares) and C1 (empty squares). At the bottom is a table that reports the characterizations and IC₅₀ of each compound.

FIG. 31 shows the results of a virucidal assay of dendritic polyglycerols 2 and R17. Both show strong virucidal activity.

FIG. 32 shows on the top, dose-response curve of dendritic polyglycerols RP3, RP3B, RN4, RN4B. (The compounds RP32 and RN42 are mentioned in the dose-response curve, having been tested at the same time, but do not form part of the disclosure.) At the bottom are shown the virucidal assay results for RP3B and RNB, showing that these compounds have virustatic (reversible) but not virucidal activity. The table at the bottom summarizes the characterizations and IC₅₀ for RP3, RP3B, RN4 and RN4B.

FIG. 33 shows on the top, a dose-response curve for dendritic polyglycerols R21 and RX. At the bottom are shown the virucidal assay results for both compounds, showing virucidal activity. The table at the bottom summarizes their characterizations and IC₅₀.

FIG. 34 shows on the top, a dose-response curve and the cell viability of dendritic polyglycerol RX. At the bottom, results of the virucidal assay that shows virucidal activity. A table on top recaps the data.

FIG. 35 shows on the top, the dose-response curve of dendritic polyglycerol R19B. At the bottom, a table reports the characterizations and IC₅₀ for RX and R19B.

FIG. 36 shows a comparison of the antiviral activity of MUS:OT gold nanoparticles (solid circles) MUS-CD (solid squares) and RX (solid triangles), on the top in concentration (log of ug/ml) and at the bottom in molarity (log of nM). In both cases RX outperforms the other two compounds.

FIG. 37 shows the hydrodynamic diameter measured by DLS (dynamic light scattering) of the non-functionalized core dPG (C1) in phosphate buffer at concentration 1 mg/mL. The results are for three measurements.

FIG. 38 shows gel permeation chromatography (GPC) diagram of the non-functionalized core dPG (C1). (Mn:7.2 kDa, Mw: 10 kDa, PDI: 1.4).

FIG. 39 shows the ¹HNMR spectra of compounds (2), (R17) and (R18).

FIG. 40 shows cytotoxicity data for compounds (RX), (R17) and (R21).

### DETAILED DESCRIPTION OF THE DISCLOSURE

### Definitions

As used in the present specification, the following words and phrases are generally intended to have the meanings as set forth below, except to the extent that the context in which they are used indicates otherwise. In the case of conflict, the present specification, including definitions, will control. Unless defined otherwise, all technical and scientific terms used herein have the same meaning as is commonly understood by one of skill in art to which the subject matter herein belongs. As used herein, the following abbreviations and definitions are supplied in order to facilitate the understanding of the present disclosure.

As used in the specification and claims, the singular form “a”, “an” and “the” includes plural references unless the context clearly dictates otherwise.

The term “about” as used in conjunction with a number or a range of numbers, indicates that such number/range will be understood to be approximate. Thus, “about 2” encompasses the integers 1, 2, 3 and 4. The term “about 5 to 30” should be read as “about 5 to about 30” and encompasses, e.g., ranges from 4 to 32, 4 to 28, 6 to 33 and 3 to 27.

As used herein the term “alkene” or “alkenyl” refers to a monoradical branched or unbranched, unsaturated or polyunsaturated hydrocarbon chain, having from about 2 to 30 carbon atoms, more particularly about 5 to 30 carbon atoms and still more particularly about 7 to about 15 carbon atoms. This term is exemplified by groups such as ethenyl, but-2-enyl, hex-2,5-dienyl, (2E,6E)-5-methyl-9λ³-nona-2,6-diene and the like. The term “alkenyl” when recited to specify a group linking to another moiety [such as carboxyalkenyl or —(C₅ to C₃₀ alkene)-COOH] refers to a diradical branched or unbranched, unsaturated or polyunsaturated hydrocarbon chain consisting of an alkenyl monoradical, a terminal hydrogen of which is substituted by such other moiety.

As used herein, the term “alkyl” refers to a monoradical branched or unbranched saturated hydrocarbon chain containing from 1 to 50 carbon atoms, particularly 5 to 30 carbon atoms. Representative examples of alkyl include, but are not limited to methyl, ethyl, n-propyl, iso-propyl, n-butyl, iso-butyl, n-pentyl, n-hexyl, n-heptyl, n-octyl, n-nonyl, n-decyl and 6-isopropyl-3-methyl-10λ³-decane. The term “alkyl” when recited to specify a group linking to another moiety (such as carboxyalkyl) refers to a diradical branched or unbranched saturated hydrocarbon chain derived from an alkyl monoradical, a terminal hydrogen of which is substituted by such other moiety; exemplified by groups such as methylene (—CH₂—), ethylene (—CH₂CH₂—), propylene isomers [e.g., —CH₂CH₂CH₂— and —CH(CH₃)—CH₂—] and the like. The term “substituted alkyl” refers to an alkyl group in which 1 or more (up to about 5, particularly up to about 3) hydrogen atoms is/are independently replaced by a substituent selected from the group comprising: alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkylamidoalkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthio alkyl, alkylthioalkoxy, alkynyl, alkynyloxy, alkynylthio, aryl, arylcarbonyl, aryloxy, arylsulfonyl, carboxy, carboxyalkyl, carboxyalkoxy, cyano, cyanoalkoxy, cyanoalkyl, cyanoalkylthio, 1,3-dioxolanyl, dioxanyl, dithianyl, ethylenedioxy, formyl, formylalkoxy, formylalkyl, haloalkenyl, haloalkenyloxy, haloalkoxy, haloalkyl, haloalkynyl, haloalkynyloxy, halogen, heterocycle, heterocyclocarbonyl, heterocycloxy, heterocyclosulfonyl, hydroxy, hydroxyalkoxy, hydroxyalkyl, mercapto, mercapto alkoxy, mercapto alkyl, methylenedioxy, nitro, sulfinyl and sulfonyl. Particular substiituents for “substituted alkyl” are selected from the group comprising: alkenyl, alkenylthio, alkoxysulfonyl, alkylcarbonylthio, alkylsulfinyl, alkylsulfinylalkyl, alkyl sulfonyl, alkylsulfonylalkyl, alkylthio, alkylthioalkoxy, alkynylthio, aryl, arylcarbonyl, aryloxy, arylsulfonyl, cyanoalkylthio, dithianyl, heterocyclosulfonyl, hydroxy, hydroxyalkoxy, hydroxyalkyl, mercapto, mercapto alkoxy, and mercapto alkyl.

As used herein, the term “and/or” used in a phrase such as “A and/or B” herein is intended to include “A and B”, “A or B”, “A”, and “B”.

As used in the specification and claims, the term “at least one” used in a phrase such as “at least one C atom” can mean “one C atom” or “two C atoms” or more than two C atoms.

As used herein, the terms “carboxylate”, “carboxy”, “carboxyl” and “carboxylic” refer to the moiety “—C(O)OH”, which can be written, interchangeably, as “—COOH” and “—COO⁻”, and should be read to include pharmaceutically acceptable salts, such as “—COO⁻Na⁺”. The term also applies to such moieties attached to a hydrocarbon linker, for example a C₁₋₅₀ alkyl group as defined herein, to form a carboxyalkyl moiety.

The term “comprise” is generally used in the sense of include, that is to say permitting the presence of one or more features or components. Also as used in the specification and claims, the language “comprising” can include analogous embodiments, as contrasted with the terms “consisting of” which includes only the embodiment recited and “consisting essentially of” which includes analogous embodiments to the extent that they do not materially affect the basic and novel characteristics of the claimed subject matter. As used herein, the term “degree of functionalization” can be abbreviated as “DF”. The term refers to the number of R functional groups bearing a moiety selected from the group: -(optionally substituted C₅ to C₃₀ alkyl)-COOH, —(C₅ to C₃₀ alkene)-COOH, —(CH₂)_{z}—O—(CH₂)_{y}—COOH, —(CH₂)_{z}—S—(CH₂)_{y}—COOH, -(optionally substituted C₅ to C₃₀ alkyl)-SO₃⁻, —(C₅ to C₃₀ alkenyl)-SO₃⁻, -(optionally substituted C₅ to C₃₀ alkyl)-OSO₃, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻ and —(CH₂)_{z}—S—(CH₂)_{y}—OSO₃⁻ determined as a percentage of all of the free hydroxyl groups in a given dendritic polyglycerol. The DF for the dendritic polyglycerols of the disclosure is at least 30% (i.e., between 30% and 100%) or particularly between about 30% and 90%, more particularly between about 40% and 75%, or about 50%. Degree of functionalization can be measured by ¹HNMR, as described in greater detail below. It will be appreciated by those skilled in the art that some, but not all of the free hydroxyl groups within the dPGC will be functionalized in the syntheses described below and bear the functional moiety represented by R and that it is not feasible to assign the specific location of a particular functional group as existing at one or the other of the two groups shown as R in Formula I. Such functionalization within the dPGC will diminish proceeding inward from the more accessible periphery to the less accessible center of the core, but will be inherently counted in the degree of functionalization measurement by ¹HNMR.

As used herein, the term “dendrimer” refers to nano-sized, synthetic, highly branched polymers and oligomers having a well-defined chemical structure that that radially, symmetrically, identically branches from an initial monomeric unit, typically forming spherical (e.g. ovoid, ellipsoid, etc. . . . ) macromolecules.

As used herein, the term “dendritic” refers to dendrimer-like highly branched polymers, copolymers or oligomers having a chemical structure resembling that of a dendrimer. Dendritic compounds have a core including a given number of generations of branches or arms, and a plurality of end groups. The branches start from an initial monomeric unit (e.g., trimethylolpropane) but are not identical, typically as the result of incomplete bonding in the early steps of polymerization. The generations of arms consist of structural monomeric units; these can be identical or incomplete for a given generation of arms (or non-identical in the case of dendritic copolymers), and can be the same as the first generation or can branch differently for subsequent generations of arms. (These monomeric units are glycerol in a dendritic polyglycerol.) The generations of arms extend radially in a geometrical progression from the initial monomeric unit until the end (or N^{th}) generation (also described as the periphery). Dendritic (in the sense of dendrimer-like) includes molecules containing non-symmetrical branching. Dense star polymers, starburst polymers and rod-shaped dendrimers can be considered dendritic.

As used herein, the term “dendritic polyglycerol” or “dPG” refers to a glycerol polymer having a plurality of branch points and multifunctional branches that lead to further branching with polymer growth. Dendritic polymers can be obtained by a one-step polymerization process and form a polydisperse system with varying degrees of branching. FIG. 1 illustrates the structure and a method for synthesizing a dPG. Methods of making a variety of such polymers are known in the art and further described herein.

As used herein, the term “dendritic polyglycerol core” or “dPGC” refers to an entire dendritic polyglycerol serving as a substrate for functionalization.

As used herein, the term “functionalized” means having chemically bound substituent groups, also referred to as functional groups, functional moieties or functional units, such as bioactive ligands. The dendritic polyglycerols useful for the present disclosure can contain only a single functional unit per branch or can contain two of the same or different functional units per branch.

As used herein, the terms “glycerol” and “glycerine” and “glycerin” all refer to the monomeric unit propane 1,2,3-triol.

As used herein, the term “hyperbranched” as in “hyperbranched polymer” or “HBP” or “hBP” is used synonymously with “dendritic) when it refers to a polymer or oligomer that branches radially from a central core incorporating plural copies of at least one branching monomer unit. This term is not synonymous with “dendritic” in the case of linear polymers that branch following a cylindrical symmetry or any other branching macromolecule that does not follow a radial branching symmetry. In contrast, hyperbranched polymers (HBPs) incorporate monomers that have three or more reacting groups and thus result in branched polymers. HBPs can be homopolymers composed of a hyperbranched single monomer, or can be copolymers of branching monomers (those able to react at three or more positions) with other branching monomers or with linear monomers (those able to react at only two positions). The HBP compounds employed herein typically are considered to be biocompatible or pharmaceutically acceptable polymers, such that they are suitable for administration to human and/or veterinary subjects. Certain disclosed embodiments of the HBP, e.g., the hyperbranched polyglycerol (hPG) polymer are homopolymers that contain only repeating glycerol subunits. In another example, the HBP can be a heteropolymer that includes one, two or more other polymer subunits. HBPs are well known in the art (see, e.g. Gao and Yan, Prog. Polym. Sci. 29 (2004) 183-275). Examples of HBP compounds, methods of synthesizing them using, for example, a single monomer methodology and double-monomer methodology, modifying, and functionalizing the compounds are disclosed herein and in Macromolecules 1999, 32, 4240-4246 (polyglycerol) and in Biomaterials 2006, 27:5471-5479, and Gao and Yan, Prog. Polym. Sci. 29 (2004) 183-275.

As used herein, the term “mammal” (for purposes of treatment) refers to any animal classified as a mammal, including humans, domestic and farm animals or pet animals, such as dogs, horses, cats, cows, monkeys etc. Particularly, the mammal is human.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined. It will be understood by those skilled in the art with respect to any group containing one or more substituents that such groups are not intended to introduce any substitution or substitution patterns (e.g., substituted alkyl including optionally substituted cycloalkyl groups, which in turn are defined as including optionally substituted alkyl groups, potentially ad infinitum) that are sterically impractical and/or synthetically non-feasible.

The term “pharmaceutically acceptable ester” refers to esters of the compounds of the present disclosure, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates, ethylsuccinates, morpholinoethyl esters and the like.

The term “pharmaceutically acceptable salts” as used herein refers to salts that retain the desired biological activity of the compounds the disclosure and includes pharmaceutically acceptable acid addition salts and base addition salts. Suitable pharmaceutically acceptable acid addition salts of the compounds of Formula I may be prepared from an inorganic acid or from an organic acid, or can be prepared in situ during the final isolation and purification of the compounds of the disclosure. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable pharmaceutically acceptable base addition salts of the compounds of Formula I include metallic salts made from lithium, sodium, potassium, magnesium, calcium, aluminium, and zinc, and organic salts made from organic bases such as choline, diethanolamine, morpholine. Other examples of organic salts are: ammonium salts, quaternary salts such as tetramethylammonium salt; amino acid addition salts such as salts with glycine and arginine. Additional information on pharmaceutically acceptable salts can be found in Remington's Pharmaceutical Sciences, 19th Edition, Mack Publishing Co., Easton, Pa. 1995. In an embodiment, the pharmaceutically acceptable salt of the compounds of the disclosure is a sodium salt.

As used herein the terms “subject” or “patient” are well-recognized in the art, and, are used interchangeably herein to refer to a mammal, including dog, cat, rat, mouse, monkey, cow, horse, goat, sheep, pig, camel, and, most particularly, a human, and other, e.g., avian animals, such as a chicken. In certain embodiments, the terms “subject” or “patient” refer to a human and animals, such as dog, cat, rat, mouse, monkey, cow, horse, goat, sheep, pig, camel, chicken. In some embodiments, the subject is a subject in need of treatment, or a subject being infected by a virus. In other embodiment, a subject can be an animal infected by a virus, such as a chicken. However, in other embodiments, the subject can be a healthy subject or a subject who has already undergone treatment. The term does not denote a particular age or sex. Thus, adult, children and newborn subjects, whether male or female, are intended to be covered.

As used herein, the term “sulfate” refers to a group written, interchangeably, as —O—SO₃⁻, —O—SO₃H, —SO₄²⁻ or —SO₄H₂ and includes groups attached to a hydrocarbon linker, for example a C₁₋₅₀ alkyl group as defined herein, to form an “alkylsulfate”, particularly C₄₋₂₀ alkylsulfates.

As used herein, the term “sulfonate” refers to a group written, interchangeably, as —SO₃⁻ or —SO₃H and includes groups attached to a hydrocarbon linker, for example a C₁₋₅₀ alkyl group as defined herein, to form an “alkylsulfonate”, particularly C₄₋₂₀ alkylsulfonates.

As used herein, the term “therapeutically effective amount” refers to an amount of a compound of the disclosure effective to alter a virus, and to render it inert, in a recipient subject, and/or if its presence results in a detectable change in the physiology of a recipient subject, for example ameliorates at least one symptom associated with a viral infection, prevents or reduces the rate transmission of at least one viral agent.

As used herein, the term “treatment” refers to both therapeutic treatment and prophylactic or preventative measures. Those in need of treatment include those already being infected by a virus, as well as those in which the viral infection is to be prevented or those who are likely to come into contact with a virus. Hence, the mammal, particularly human, to be treated herein may have been diagnosed as being infected by a virus, or may be predisposed or susceptible to be infected by a virus. Treatment includes ameliorating at least one symptom of, curing and/or preventing the development of a disease or condition due to the viral infection. Preventing is meant attenuating or reducing the ability of a virus to cause infection or disease, for example by affecting a post-entry viral event.

As used herein, the term “virucidal” refers to a characterization of antiviral efficacy determined by in vitro testing demonstrating irreversible inhibition of the infectivity of a virus following interaction with an antiviral compound or composition. Even following termination of the interaction (for example, by dilution) and absent any added materials or conditions promoting viral reconstitution, it is essentially impossible for the virus to resume infectivity. Interaction with antiviral compound or composition alters the virus, rendering it inert, and thereby prevents further infections.

As used herein, the term “virustatic” refers to a characterization of antiviral efficacy determined by in vitro testing demonstrating reversible inhibition of the infectivity of a virus following interaction with an antiviral compound or composition. Once the interaction terminates (for example, by dilution) and absent any added materials or conditions promoting viral reconstitution, it is possible for the virus to resume infectivity.

### Compounds of the Present Disclosure

A biomimetic strategy has been employed to develop broad-spectrum virucidal drugs. To limit toxicity, it has been decided to stay away from known bio-toxic approaches and to concentrate on mimicking cell-receptors, so to strongly attach to their corresponding viral ligand and generate local viral deformation that would ultimately lead to irreversible viral damage, possibly to viral disassembly. To achieve broad-spectrum efficacy, it was aimed at virus-cell interactions that are common to many viruses. One of these interactions is that between viruses and cell-surface attachment receptors that represent the very first step of the virus replication cycle. Many viruses, including HIV-1, HSV, HCMV, HPV, Respiratory syncytial virus (RSV) and filoviruses, exploit heparan sulfate proteoglycans (HSPGs) as attachment receptors, as HSPGs are expressed on the surface of almost all eukaryotic cell types. The binding between viruses and HSPGs usually occurs via the interaction of stretches of basic amino acids on viral proteins (basic domains) with the negatively charged sulfated groups of heparan sulfate (HS) chains on the glycocalix of the cell surface.

An aspect of the present disclosure provides a novel class of virucidal compounds designed to mimic cell surface sugars and have very low toxicity as well as broad-spectrum activity against HSV-2 and other HSPG-seeking viruses at nanomolar concentration with a virucidal effect (i.e. the ability of irreversibly inhibiting viral infectivity). The virucidal compounds of the disclosure have been prepared by partial or complete functionalization of dendritic polyglycerols (dPG), as a soft biocompatible platform, with different sufficiently long ligands having functional groups, such as sulfate and sulfonate. dPG is a highly branched polymer with a flexible polyether backbone and high density of surface hydroxyl groups that can be further modified with different ligands.

The present disclosure provides certain dendritic polyglycerol compounds. The compositions are antivirals, have proved to be virucidal inhibitors of SARS-CoV-2, and can be used to treat COVID-19 and other viral diseases.

The context in which certain compounds of the disclosure were discovered is significant, in that the first carboxyalkyl functionalized dendritic polyglycerol was synthesized to be employed as a negative control in testing the utility of other compounds for inhibition of SARS-CoV-2. It was believed that the cell surface receptors sought by the virus did not comprise —COO⁻ groups. When tested together with other promising anti-SARS-CoV-2 candidate molecules, it was surprisingly discovered that the carboxyalkyl dendritic polyglycerol indeed has virucidal activity.

Accordingly, the present disclosure relates to the compounds, pharmaceutical formulations, methods of treatment employing such compositions, as represented by Formula I:

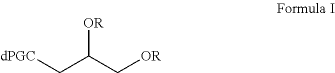

wherein:
dPGC is dendritic polyglycerol core in which OR represents the free OH groups within and at the peripheryu of the core, having an average molecular weight from 5 to 100 kDa as measured by GPC;

R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH, —SO₃⁻, —(CH₂)_{z}—S—(CH₂)_{y}—CH₃, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃⁻, —(C₅ to C₃₀ alkenyl)-SO₃⁻, -(optionally substituted C₅ to C₃₀ alkyl)-O—SO₃⁻, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃⁻;

y is an integer from about 4 to about 30;

z is an integer from 1 or 2 to about 20;

y+z is an integer from about 5 or 6 to about 30; and

having a DF of at least about 30%,

or a pharmaceutically acceptable salt or pharmaceutically acceptable ester.

In one aspect, the disclosure provides a compound, pharmaceutically acceptable salt or pharmaceutically acceptable ester of Formula I wherein:

dPGC is dendritic polyglycerol core having an average molecular weight from 5 to 100 kDa as measured by GPC;

R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH;

y is an integer from about 4 to about 30;

z is an integer from 1 to about 20;

y+z is an integer from about 5 to about 30; and

having a DF of at least about 30% as measured by ¹HNMR, where R contributing to the DF has a —COOH.

In one aspect, R is functionalized with a carboxylic acid bearing aliphatic chain, e.g., having from about 6 to about 20 carbon atoms, and alternatively from about 8 to about 15 carbon atoms. The variable “y” can be an integer from about 4 to about 20, e.g., from about 8 to about 13 and alternatively about 11. The variable “z” is an integer from about 2 to about 10, e.g., from about 2 to about 5, and alternatively about 3. The sum “y+z” is an integer from about 10 to about 16, e.g., about 14.

Alternatively, in Formula I,

dPGC is dendritic polyglycerol core having an average molecular weight from 5 to 100 kDa as measured by GPC;

R can be the same or different and is selected from the group comprising: —H, —SO₃⁻, —(CH₂)_{z}—S—(CH₂)_{y}—CH₃, -optionally substituted C₅ to C₃₀ alkyl, —C₃ to C₃₀ alkenyl, C₅ to C₃₀ ω-hydroxyalkyl, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃⁻, —(C₅ to C₃₀ alkenyl)-SO₃⁻, -(optionally substituted C₅ to C₃₀ alkyl)-OSO₃, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃;

y is an integer from about 4 to about 30;

z is an integer from about 2 to about 20;

y+z is an integer from about 6 to about 30; and

having a DF of at least about 30% as measured by 1 HNMR, where R contributing to the DF has an —SO₃⁻ or an —O—SO₃⁻.

R can be functionalized with a sulfate- or sulfonate-bearing aliphatic chain, e.g., having from about 6 to about 20 carbon atoms, and alternatively from about 8 to about 15 carbon atoms. The variable “y” can be an integer from about 4 to about 20, e.g., from about 8 to about 13 and alternatively about 11. The variable “z” can be an integer from about 2 to about 10, e.g., from about 2 to about 5, and alternatively about 3. The sum “y+z” can be an integer from about 10 to about 16, e.g., about 14.

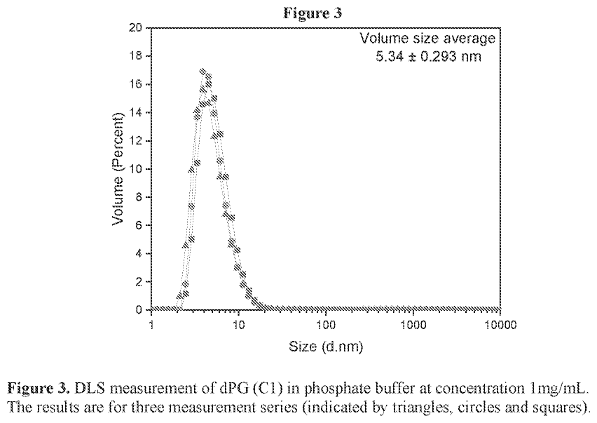

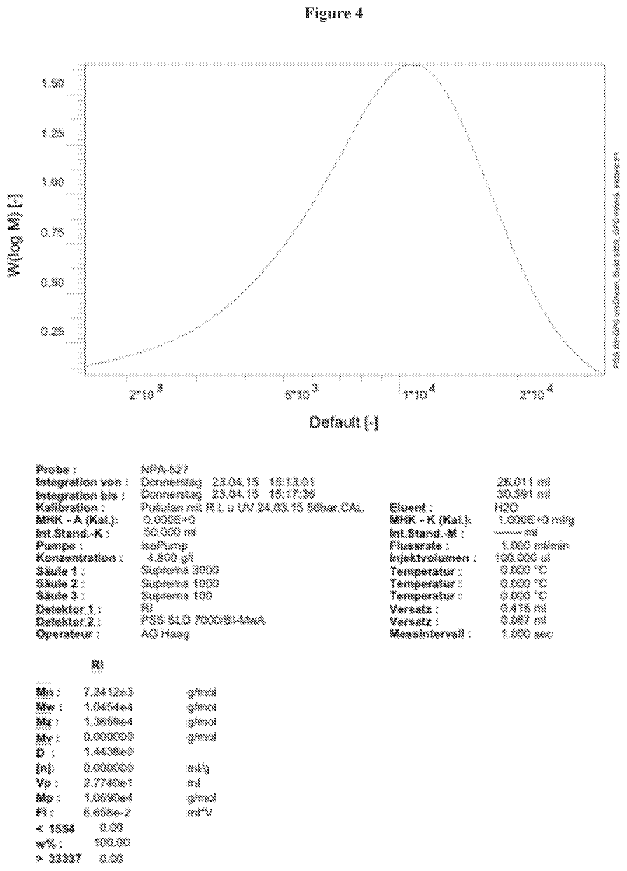

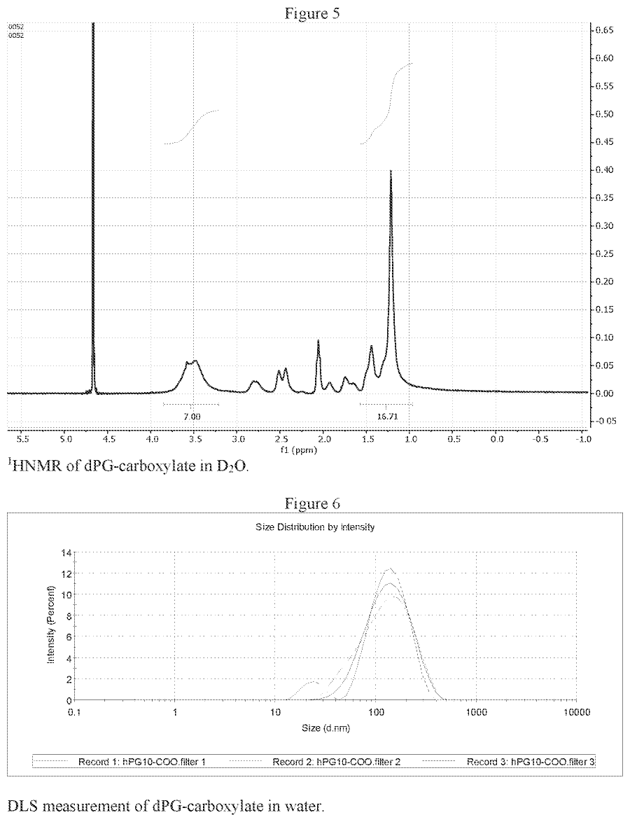

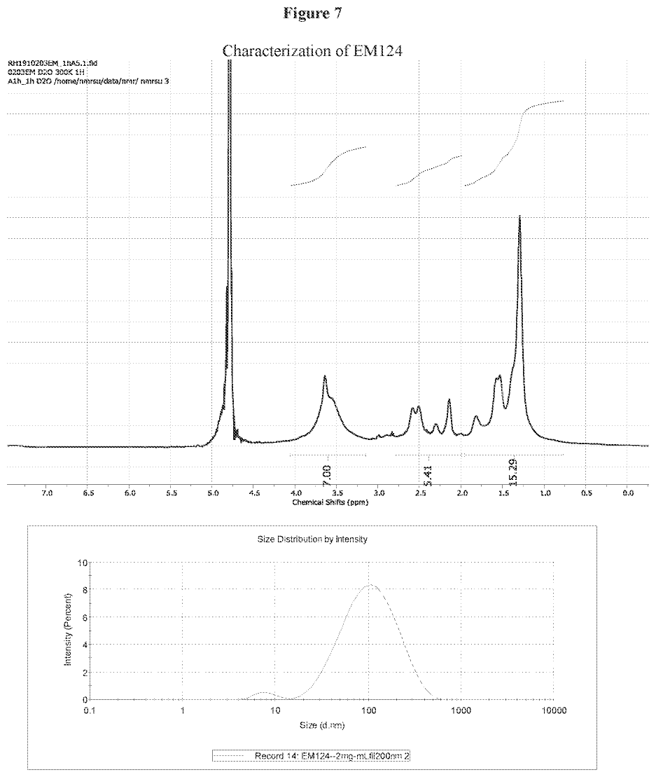

The dendritic polyglycerol core (dPGC) according to the disclosure has a size from about 4 to 15 nm and a molecular weight from about 5 to 100 kDa. It is composed of repeated units of glycerine with the formula (RO—CH₂)₂CH—OR wherein R=H or an adjacent glycerine unit on a multifunctional polyhydroxy starter molecule having a plurality of OH groups, for example 2 to 4 OH groups, such as 2-ethyl-2-(hydroxymethyl)propane-1,3-diol. FIG. 1 shows a representative example of a dPG formed from 2-ethyl-2-(hydroxymethyl)propane-1,3-diol polymerized with glycerol monomers. One particular dPGC identified as “(C1)” has a hydrodynamic diameter (size) of 5.34±0.293 nm and molecular weight=>GPC (H₂O) Mₙ=7.2 kDa, M_{w}=10.4 kDa. FIGS. 3 and 4 show the results of dynamic light scattering (DLS) characterizing the size and gel permeation chromatography (GPC) characterizing the molecular weight of dendritic polyglycerol core (C1). It should be noted that the weight and size of functionalized dPG products of Formula I will be greater than the weight and size of the core (dPGC) that was functionalized.

In one aspect of the disclosure, the R substituents that contribute to DF are selected from the group comprising: -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH. In another aspect, the R substituents that contribute to DF are selected from the group comprising: -(optionally substituted C₅ to C₃₀ alkyl)-COOH, —(CH₂)_{z}—O—(CH₂)_{y}—COOH, and —(CH₂)_{z}—S—(CH₂)_{y}—COOH.

In one aspect of the disclosure, the R groups that contribute to DF are selected from the group comprising: -(optionally substituted C₅ to C₃₀ alkyl)-SO₃⁻, —(C₅ to C₃₀ alkenyl)-SO₃⁻, -(optionally substituted C₅ to C₃₀ alkyl)-OSO₃⁻, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃ and —(CH₂)_{z}—S—(CH₂)_{y}—OSO₃⁻. In another aspect, particularly the compositions of matter, the R groups that contribute to DF are selected from the group comprising: —(C₅ to C₃₀ alkenyl)-SO₃⁻, (C₅ to C₃₀ alkenyl)-OSO₃, —(CH₂)_{z}—O—(CH₂)_{y}—SO₃⁻, —(CH₂)_{z}—O—(CH₂)_{y}—OSO₃⁻, —(CH₂)_{z}—S—(CH₂)_{y}—SO₃ and —(CH₂)_{z}—S—(CH₂)_{y}—OSO₃⁻. In yet another aspect, the R groups that contribute to DF are selected from the group comprising: —(CH₂)_{z}—O—(CH₂)_{y}—SO₃, —(CH₂)_{z}—O—(CH₂)_{y}—O—SO₃, —(CH₂)_{z}—S—(CH₂)_{y}—SO₃ and —(CH₂)_{z}—S—(CH₂)_{y}—OSO₃⁻.

According to an embodiment, the present disclosure provides a compound of Formula I comprising a dendric polyglycerol core as defined above functionalized with a plurality of same or different R substituents as defined above that are bound to the core, provided however, that not each of said R substituents necessarily comprises a COOH, a sulfate or a sulfonate group, and wherein the degree of functionalization is as defined above. R will comprise a hydrogen or an incompletely reacted precursor group when not fully functionalized with a COOH, sulfate or sulfonate-bearing aliphatic chain. Incompletely reacted precursor groups for R can include, without limitation: H, -optionally substituted C₅ to C₃₀ hydroxyalkyl, —C₃ to C₃₀ alkenyl (particularly —CH₂—CH═CH₂), —SO₃⁻, C₅ to C₃₀ ω-hydroxyalkyl, C₅ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl.

Another aspect of the present disclosure provides a compound of Formula II

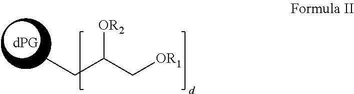

wherein:

dPGC is a dendritic polyglycerol core having a size from 4 to 200 nm and a molecular weight from 5 to 100 kDa,

each R₁, independently, is optionally substituted alkyl-based ligand selected from the group comprising —(CH₂)_{y}—CH₃, —(CH₂)_{y}—SO₃H, —(CH₂)_{y}—OSO₃H, —(CH₂)₃—S—(CH₂)_{y}—OSO₃H. In some embodiments, each R₁, independently, is optionally substituted alkyl-based ligand selected from the group comprising —(CH₂)_{y}—SO₃H, —(CH₂)_{y}—OSO₃H, —(CH₂)₃—S—(CH₂)_{y}—SO₃H, —(CH₂)₃—S—(CH₂)_{y}—OSO₃H. In other embodiments, each R₁, independently, is optionally substituted alkyl-based ligand selected from the group comprising —(CH₂)_{y}—CH₃, —(CH₂)₃—S—(CH₂)_{y}—SO₃H, —(CH₂)₃—S—(CH₂)_{y}—OSO₃H. In further embodiments, at least one R₁ is not —(CH₂)_{y}—SO₃H and/or —(CH₂)_{y}—OSO₃H. In some other embodiments, each R₁, independently, is optionally substituted alkyl-based ligand selected from the group comprising —(CH₂)_{y}—CH₃, —(CH₂)_{y}—SO₃H, —(CH₂)_{y}—OSO₃H, —(CH₂)₃—S—(CH₂)_{y}—OSO₃H, provided that at least one R₁ is not —(CH₂)_{y}—SO₃H and/or —(CH₂)_{y}—OSO₃H.

each R₂, independently, is —H, —SO₃H, —CH₂—CH═CH₂, —(CH₂)₃—S—(CH₂)₂—CH₃. In some embodiments, each R₂, independently, is —H or —SO₃H. In other embodiments, each R₂, independently is —H, —CH₂—CH═CH₂, —(CH₂)₃—S—(CH₂)₂—CH₃. In other embodiments, each R₂, independently is —CH₂—CH═CH₂, —(CH₂)₃—S—(CH₂)₂—CH₃. In further embodiments, at least one R₂ is not —H or —SO₃H. In other embodiments, each R₂, independently, is —H, —SO₃H, —CH₂—CH═CH₂, —(CH₂)₃—S—(CH₂)₂—CH₃, provided that at least one R₂ is not —H or —SO₃H.

y is at least 4, particularly from 4 to 30 or from 8 to 20, most particularly y is 7 to 11 or most particularly y is 11. In other embodiments, y is at least 5, at least 6, at least 7, at least 8, at least 9, at least 10, at least 11. In other embodiments, y is at maximum 100, at maximum 70, at maximum 50, at maximum 40, at maximum 30, at maximum 25, at maximum 20, at maximum 15

and having a degree of functionalization (d) that is at least 30%, at least 31%, at least 35%, at least 40%, or at least 45%; at maximum 100%, at maximum 95%, or at maximum 90%; particularly from 30% to 100%, from 31% to 100%, from 35% to 100% from 40% to 100%, from 30% to 90%, 31% to 90%, 35% to 90% or 40% to 90%, or a pharmaceutically acceptable salt thereof.

### Nomenclature

In the present specification, the dendritic polyglycerols are named using the following format: The subscript following “dPG” indicates the weight of the dendritic polyglycerol being named, e.g., dPG_{10 kDa} names a dendritic polyglycerol having a weight of 10 kDa. The text following such weight indication identifies the functional group(s) and the subscript that follows indicates the degree of functionalization. Thus, dPG_{10 kDa}-C₃SC₁₀—COO⁻Na⁺_{100%} names a dendritic polyglycerol having the structure shown below in Formula dPG1:

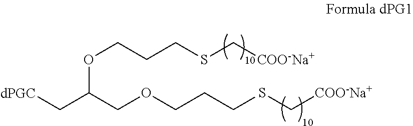

which is a compound of Formula 1 where the dPG core has a weight of 10 kDa, R is —(CH₂)_{z}—S—(CH₂)_{y}—COO⁻Na⁺ where z is 3 and y is 10, with a DF of 100%.

Similarly, dPG_{10 kDa}-C₁₁-sulfate_{50%}/sulfonate_{50%} names a dendritic polyglycerol having the structure shown below in Formula RX:

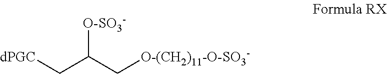

which is a compound of Formula 1 where the dPG core has a weight of 10 kDa, where about half of the free hydroxyls are undecane sulfate with a DF of 50%, and the other half are a sulfonate with a DF of 50%.

### Synthesis of the Compounds of Formula I

Syntheses of the compounds of Formula I are described below with reference to the Reaction Schemes.

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform, methylene chloride (or dichloromethane), diethyl ether, methanol, pyridine and the like]. Unless specified to the contrary the solvents used in the reactions of the present disclosure are inert organic solvents. Reactions take place at room temperature and 1 atmosphere of pressure unless otherwise indicated.

Isolation and purification of the compounds and intermediates described herein can be effected, if desired, by any suitable separation or purification procedure such as, for example, filtration, extraction, crystallization, column chromatography, thick-layer chromatography, or a combination of these procedures. Specific illustrations of suitable separation and isolation procedures can be had by reference to the examples hereinbelow. However, other equivalent separation or isolation procedures can, of course, also be used.

### Starting Materials

The starting materials, such as the glycerol (1) are commercially available or can be readily prepared by those skilled in the art using commonly employed synthetic methodology.

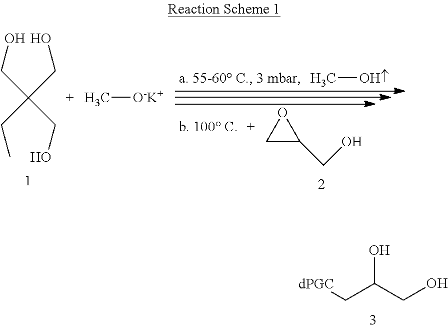

Preparation of dendritic polyglycerol: The dPG cores used in the disclosure can be synthesized according to procedures described in the literature, e.g., Sunder, A.; Frey, H.; Muelhaupt, R. Macromol Symp 2000, 153, 187.

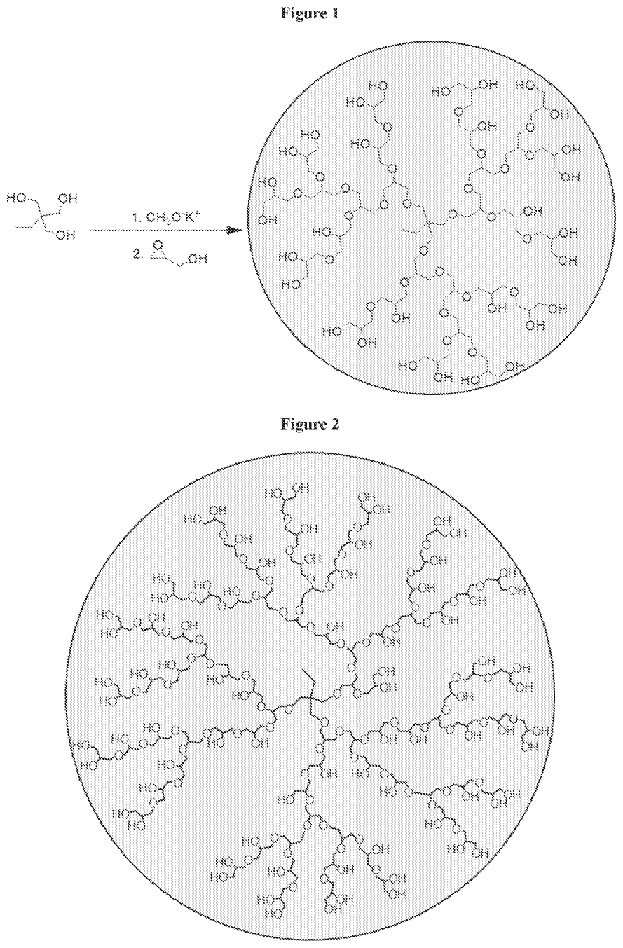

As illustrated above in Reaction Scheme 1, (a.) a glycerol such as trimethylolpropane (“TMP”) (1) is deprotonated with about 0.4 molar equivalents (“eq.”) of potassium methoxide solution (e.g., in methanol). The methanol is evaporated at about 60° C. under vacuum (about 3 mbar). (b.) The synthesis reactor is heated to about 100° C. and an excess (e.g., number of branches desired×3 eq.) of (oxiran-2-yl)methanol (“glycidol”) (2) is added slowly, e.g., over a period of about 24 hours, providing ring opening multi-branching polymerization conditions, to afford a dendritic polyglycerol (3), such as C1, the dPG illustrated in FIG. 1, or the dPG illustrated in FIG. 2, which can be conventionally isolated and purified. The molecular weight of the resulting dPG can be controlled by adjusting the molar ratio of glycidol to TMP and the ring-opening polymerization reaction time accordingly. It will be appreciated by those skilled in the art that the resulting product will be a mixture of dPGs falling within a narrow range of molecular weights, such as 8 kDa to 12 kDa, and will have an average molecular weight such as 10 kDa.

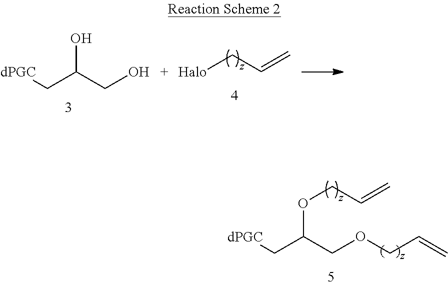

Preparation of dPGC-allyl (5): As illustrated above in Reaction Scheme 2, a dPG core (3) is functionalized in preparation for a subsequent thiol-ene click reaction, by converting the free hydroxyl groups to allyl groups through reaction with an allyl halide (e.g., bromide) (4) where z can be 0 to about 18. The dPG is dissolved in a suitable solvent (e.g., DMF) and the reaction takes place over about 12 hours; it is performed in dry condition in presence of NaH as base for deprotonation of hydroxyl groups. The resulting allyl-functionalized product (5) is conventionally isolated and purified (e.g., solvent removed under vacuum and purification by dialysis in MeOH for 2 days). The degree of functionalization (DF) can be controlled by adjusting the ratio of allyl halide to dPG, the amount of NaH, the reaction time and/or conditions, and is confirmed by 1H NMR. For example, by limiting the equivalents of allyl halide and NaH, the product corresponding to (5) where more of the groups corresponding to R remain hydrogen can be obtained.

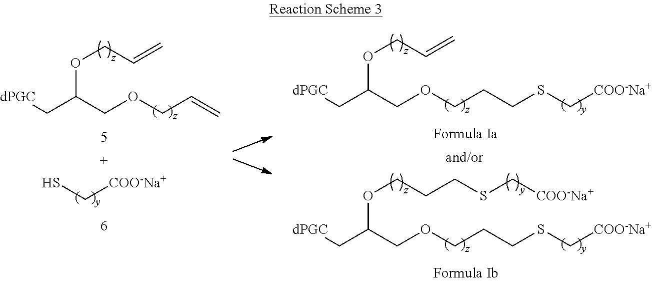

Preparation of Formulae Ia and Ib: As illustrated above in Reaction Scheme 3, dPG-di-allyl (5) (DF=100) (where z can be 0 to about 18) and about 2 eq. of an ω-mercaptoalkyl carboxylic acid sodium salt (6) (where y is an integer from about 4 to about 30) are dissolved in suitable solvent (e.g., methanol). To this solution are added 2,2-dimethoxy-2-phenylacetophenone (DMPA) as radical initiator and a catalytic amount of tris(2-carboxyethyl)phosphine hydrochloride (TCEP-HCl) (to avoid oxidation of the thiol intermediate). A few drops of water can be added to dissolve any precipitation and obtain a clear solution. The solution is degassed by flushing argon through the reaction mixture for about 10 minutes. The reaction mixture is then stirred and irradiated with UV light using a high pressure UV lamp at room temperature for about 5 hours. The product(s) from the reaction mixture can be conventionally isolated and purified, for example being dialyzed (MWCO 2 kDa), e.g., against methanol/water mixture, to remove the TCEP.HCl, DMPA and any excess of unreacted thiol compound. The product(s) will have a DF of about 85-95%. Most of the product will be Formula Ib. The products can be conventionally separated (e.g., by gel permeation chromatography or size exclusion chromatography). The successful formation of Formula Ia and/or Formula Ib can be confirmed by ¹HNMR of pure product by correlating the aliphatic protons of ligands at 1.5-1.00 ppm with the polyglycerol backbone protons at 3.7-3.2 ppm. In addition, elemental analysis can be used for the sulfur content measurement confirming the click reaction.

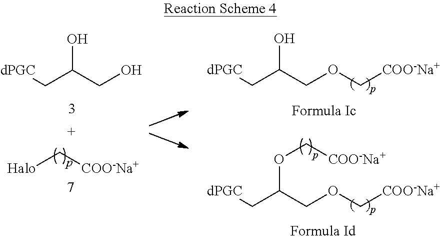

Synthesis of dPGC-carboxyalkyl: As illustrated in Reaction Scheme 4, dPG (3) is reacted with an ω-halo-alkan-1-olic acid salt (7) (about 1.5 eq.) (where p is an integer from 5 to 30) in the presence of NaH (about 2 eq.) (as a base for deprotonation of the dPG hydroxyl groups). The reaction mixture is allowed to stir for about 24 hours at about 40° C. and is then quenched by adding methanol and conventionally isolated and purified (e.g., by dialysis against methanol) to afford the corresponding dPGC-alkyl carboxylate of Formula Ic (with a DF of about 50%) or Formula Id (with a DF of about 100%) The remaining hydroxyl groups of Formula Ic can be further functionalized, for example, by sulfation.

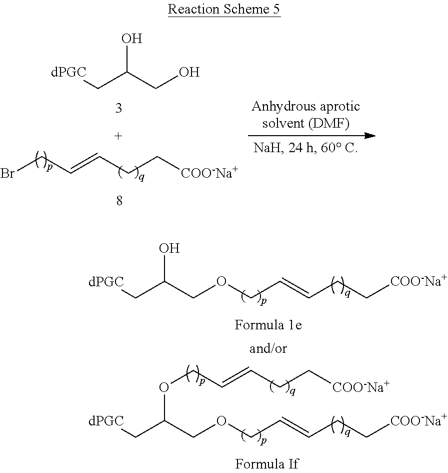

Synthesis of dPGC-R-carboxyalkene/carboxyalkene or H: As illustrated in Reaction Scheme 5, dPG (3) is reacted with an ω-bromo-alkenyl-1-carboxylic acid sodium salt (8) (where p is an integer from 1 to 28, q is an integer from 1 to 26, and p+q is an integer from 4 to 30) in the presence of NaH. The reaction mixture is allowed to stir for about 24 hours at about 60° C. to afford one or both of the corresponding alkenyl sulfonate products of Formula Ie and Formula If, which are conventionally separated (e.g., by gel permeation chromatography or size exclusion chromatography), isolated and purified.

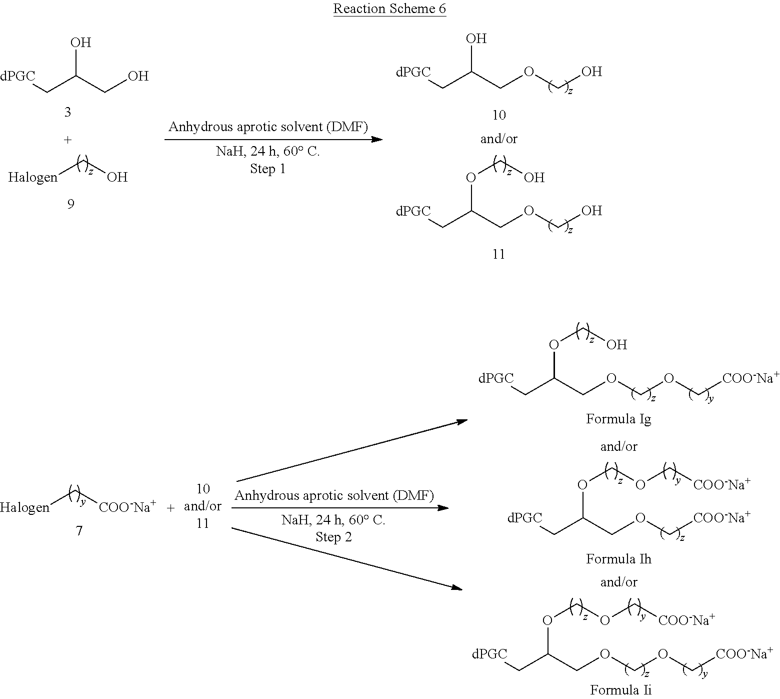

Synthesis of hydroxyalkyl dPG: As illustrated in Reaction Scheme 6, Step 1, dPG (3) is reacted with an ω-halo-alkan-1-ol (9) in the presence of NaH. The alkanol (9) is added slowly, dropwise, to minimize the formation of dimers. The reaction mixture is allowed to stir for about 24 hours at about 60° C. to afford one or both of the corresponding hydroxyalkyl products (10) and/or (11), which can be carried forward together or conventionally separated (e.g., by gel permeation chromatography or size exclusion chromatography) and carried forward independently. The products can be conventionally isolated and purified.

Synthesis of carboxyalkoxyalkyl dPGs: As illustrated in Reaction Scheme 6, Step 2, hydroxyalkyl (10) and/or (11) is/are reacted with an ω-halo-alkan-1-olic acid salt (7) in the presence of NaH. The reaction mixture is allowed to stir for about 24 hours at about 60° C. to afford one or all of the corresponding cartboxyalkyl/carboxyalkoxy products of Formula Ig, Formula Ih and/or Formula Ii, which are conventionally separated (e.g., by gel permeation chromatography or size exclusion chromatography), isolated and purified.

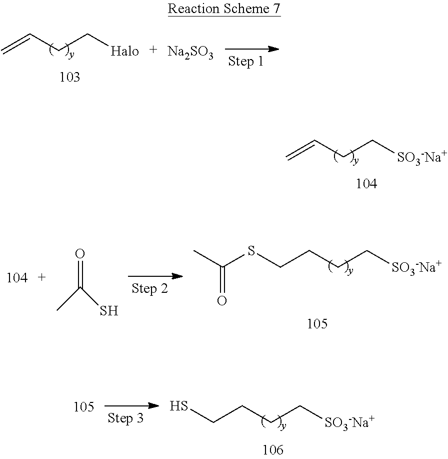

Preparation of mercapto-alkyl-sulfonate (106) As illustrated in Reaction Scheme 7, Step 1, an ω-halo-1-alkene of formula 103 where y can be 1 to 27 (e.g., where y is 6 to 12—other sizes will entail modification of the conditions) and “Halo” is bromo, chloro, fluoro or iodo, particularly bromo, is contacted with sodium sulfite (about 2 eq.) in a suitable solvent such as methanol and DI water. The mixture is refluxed (at about 102° C.) for about 48 hours to afford the corresponding ω-sulfonyl-1-alkene (104) which is conventionally isolated and purified (e.g., diethyl ether extraction, evaporation, drying and removal of inorganic salts with pure ethanol and filtration).

As illustrated in Reaction Scheme 7, Step 2, the ω-sulfonyl-1-alkene (104) obtained in Step 1 is dissolved in a suitable solvent (e.g., methanol) to afford a clear solution, removing any precipitate by filtration (to improve yield). An excess of thioacetic acid (about 2-3 eq.) is added to the solution and it is stirred in front of a UV lamp for about 12 hours. The solution is evaporated until the resulting solid residue becomes colored (e.g., orange-red), after which the solid is washed to remove the colored material (e.g., with diethyl ether) until additional colored material can no longer be removed. The resulting colored solid is dried (e.g., under high vacuum) and dissolved in a suitable solvent (e.g., methanol) to afford a colored (e.g., yellow) solution. A suitable amount of carbon black is added to the solution followed by vigorous mixing, and the mixture is filtered (e.g., through celite in fluted filter paper) to afford a clear solution, from which the solvent is completely evaporated to afford the corresponding sodium acetylthio-alkyl-1-sulfonate (105).

As illustrated in Reaction Scheme 7, Step 3, the sulfonate (105) obtained in the previous step is refluxed in 1 M HCl for 12 hours, after which the mixture is brought to pH˜3 by addition of 1 M NaOH followed by DI-water to create a volume suitable for the scale of the reaction (e.g., 1 L). The resulting solution is kept at 4° C. and crystallized over about 12 hours to yield the corresponding mercapto-alkyl sulfonate product (106) that is conventionally isolated and purified (e.g., centrifugation and dried under high vacuum). (Additional product can be extracted from the supernatant of the centrifugation step, by reducing volume and keeping it at 4° C.)

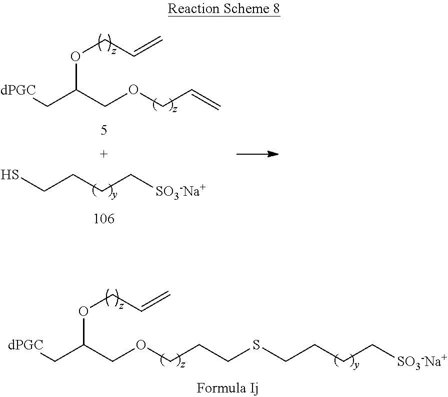

Synthesis of mercapto alkyl sulfonate functionalized dPG (Formula Ij) As illustrated in Reaction Scheme 8, a mercapto alkyl sulfonate (106) where z can be 0 to about 18 is conjugated to a dPG-allyl functionalized core (5) where y can be 1 to 27 by dissolving the reactants in a suitable solvent (e.g., water:methanol). A catalytic amount of tris(2-carboxyethyl)phosphine hydrochloride (TCEP-HCl) is added to reduce disulfide bonds and avoid oxidation of the thiol intermediate. The solution is degassed (e.g., by flushing argon through the reaction mixture for about 10 minutes) and the reaction mixture is stirred and irradiated with UV light (e.g., using a high-pressure UV lamp at room temperature) for about 6 hours. The solution is then dialyzed (MWCO 2 kDa) against water:methanol for about 2 days. The solvent is evaporated under reduced pressure and the resulting product (Formula Ia) is conventionally isolated and purified (e.g., by lyophilization). The degree of functionalization can be controlled by adjusting the ratio of mercapto alkyl sulfonate to dPG allyl and/or time of UV irradiation.

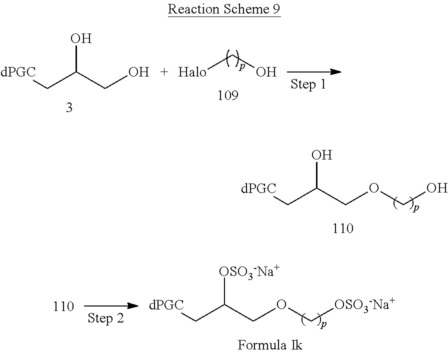

Synthesis of dPGC-R-alkanol (10) As illustrated in Reaction Scheme 9, Step 1 dPG (3) is reacted with an ω-halo-alkan-1-ol (109) (about 1.5 eq.) (where p is an integer from 5 to 30) in the presence of NaH (about 2 eq.) (as a base for deprotonation of the dPG hydroxyl groups) to obtain the corresponding dPG-Cₚ—OH with 50% of degree of functionalization. The reaction mixture is allowed to stir for about 24 hours at about 40° C. and is then quenched by adding methanol and purified by dialysis against methanol to afford the corresponding dPGC-alkanol (110).

Synthesis of dPGC-R-alkyl-sulfate/sulfate (Formula Ik) As illustrated in Reaction Scheme 9, Step 2 both hydroxyl groups of (110) are sulfated, e.g., by contact with pyridine sulfur trioxide complex or chlorosulfonic acid (about 1.5 eq.), in dry DMF at about 60° C. for about 12 hours. The reactions are quenched with water, and the pH adjusted to 8 by addition of NaOH solution. Solvent is evaporated under reduced pressure, and the product sulfated dPG (Formula Ik) is isolated and purified conventionally (e.g., dissolved in brine, dialysed with a NaCl solution, using an ever-decreasing NaCl concentration, until the medium is changed with distilled water). Degree of functionalization is determined by ¹HNMR.

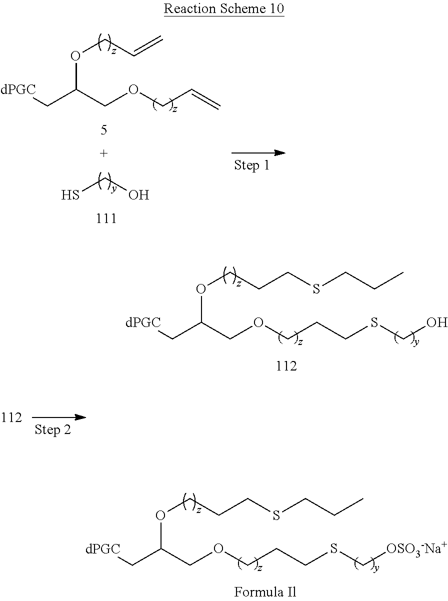

Synthesis of dPGC-R-thioalkanol/alkyl-thio-alkyl As illustrated in Reaction Scheme 10, Step 1, a dPG-allyl functionalized core (5), about 0.6 eq. of an ω-mercapto-1-alkanol (111) and a catalytic amount of 2,2-dimethoxy-2-phenylacetophenone (DMPA) (as a radical initiator) are dissolved in a suitable solvent (e.g., water:methanol). A catalytic amount of tris(2-carboxyethyl)phosphine hydrochloride (TCEP-HCl) is added (to avoid oxidation of the thiol intermediate). The solution is degassed (e.g., by flushing argon through the reaction mixture for about 10 minutes). The reaction mixture is stirred and irradiated with UV light using a high-pressure UV lamp at room temperature for about 4 hours. An excess of 1-propanthiol is added to the mixture followed by additional (e.g., about 4 hours) UV irradiation (to quench the remaining allyl group). The solution is then dialyzed (e.g., MWCO 2 kDa) against water:methanol for about 2 days. The solvent is evaporated (e.g., under reduced pressure) and the resulting intermediate (112) is carried forward in the next step.

Synthesis of dPGC-R-thioalkyl-sulfate/alkyl-thio-alkyl (Formula II) In the next step the hydroxyl group of (112) is sulfated by reaction with pyridine sulfur trioxide complex; the reaction takes place in a suitable solvent (e.g., dry DMF) at about 60° C. for about 12 hours. The reaction is then quenched with water, and the pH is adjusted to 8 (e.g., by addition of NaOH solution). The solvent is evaporated (e.g., under reduced pressure) and the product is dissolved (e.g., in brine). Dialysis is performed with a NaCl solution, using an ever-decreasing NaCl concentration, until the medium is changed with distilled water. The final product (Formula II) is obtained, e.g., after lyophilization.

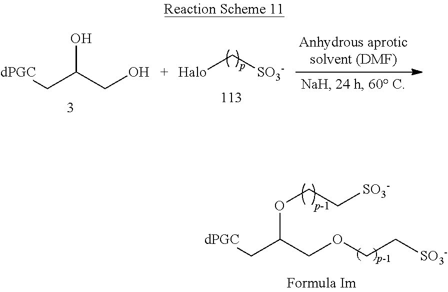

Synthesis of dPGC-R-alkyl-sulfonate/alkyl-sulfonate As illustrated in Reaction Scheme 11, dPG (3) is reacted with an ω-halo-alkyl-1-sulfonate (113) (where p is an integer from 5 to 30) in the presence of NaH. The reaction mixture is allowed to stir for about 24 hours at about 60° C. and the corresponding alkyl sulfonate product of Formula Im is conventionally isolated and purified.

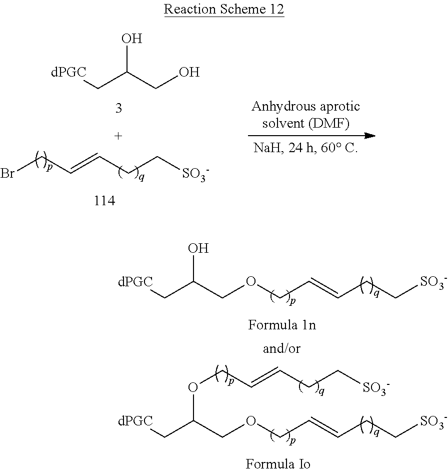

Synthesis of dPGC-Ralkenyl-sulfonate/alkyl-sulfonate or H As illustrated in Reaction Scheme 12, dPG (3) is reacted with an ω-bromo-alkenyl-1-sulfonate (114) (where p is an integer from 1 to 28, q is an integer from 1 to 26, and p+q is an integer from 4 to 30) in the presence of NaH. The reaction mixture is allowed to stir for about 24 hours at about 60° C. to afford one or both of the corresponding alkenyl sulfonate products of Formula In and Formula Io, which are conventionally separated (e.g., by gel permeation chromatography or size exclusion chromatography), isolated and purified.

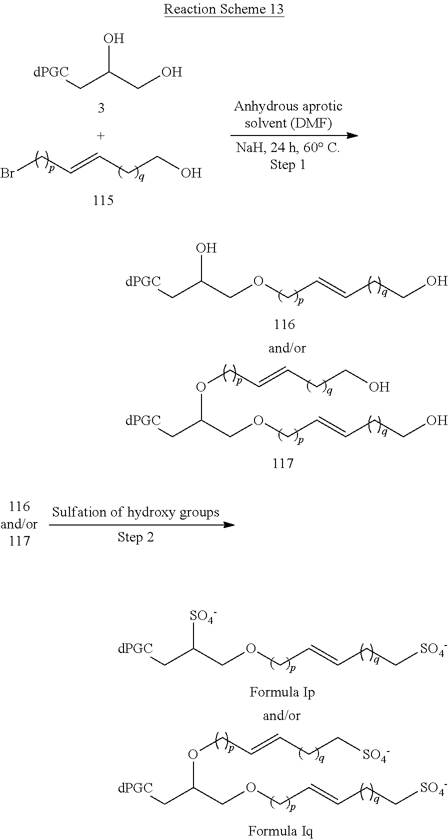

Synthesis of hydroxyalkenyl dPGs As illustrated in Reaction Scheme 13, Step 1, dPG (3) is reacted with an ω-bromo-alken-1-ol (115) (where p is an integer from 1 to 28, q is an integer from 1 to 26, and p+q is an integer from 4 to 30) in the presence of NaH. The alkenol (115) is added slowly, dropwise, to minimize the formation of dimers. The reaction mixture is allowed to stir for about 24 hours at about 60° C. to afford one or both of the corresponding hydroxyalkenyl products (116) and/or (117), which can be carried forward together or conventionally separated (e.g., by gel permeation chromatography or size exclusion chromatography) and carried forward independently. The products can be conventionally isolated and purified.

Synthesis of dPGC-R-alkenyl-sulfate/alkenyl-sulfate or H As illustrated in Reaction Scheme 13, Step 2, hydroxyalkenyl dPG (116) and/or (117) is sulfated, for example, by reaction with pyridine sulfur trioxide complex; the reaction takes place in a suitable solvent (e.g., dry DMF) at about 60° C. for about 12 hours. The reaction is then quenched with water, and the pH is adjusted to 8 (e.g., by addition of NaOH solution). The solvent is evaporated (e.g., under reduced pressure) and the product(s) is/are dissolved (e.g., in brine). Dialysis is performed with a NaCl solution, using an ever-decreasing NaCl concentration, until the medium is changed with distilled water. The final product(s) (Formula Ip and/or Formula Iq) is/are obtained, e.g., after lyophilization, conventionally separated (e.g., by gel permeation chromatography or size exclusion chromatography) if necessary, conventionally isolated and purified.

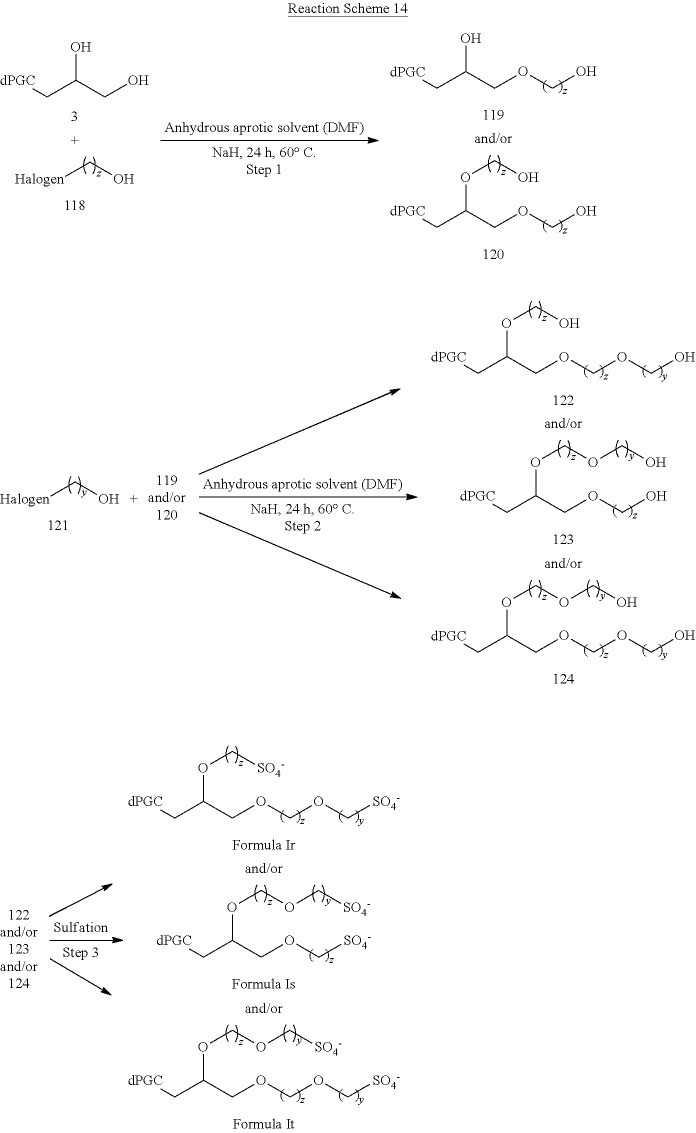

Synthesis of hydroxyalkyl dPGs As illustrated in Reaction Scheme 14, Step 1, dPG (3) is reacted with an ω-halo-alkan-1-ol (118) in the presence of NaH. The alkanol (118) is added slowly, dropwise, to minimize the formation of dimers. The reaction mixture is allowed to stir for about 24 hours at about 60° C. to afford one or both of the corresponding hydroxyalkanol products (119) and/or (120), which can be carried forward together or conventionally separated (e.g., by gel permeation chromatography or size exclusion chromatography) and carried forward independently. The products can be conventionally isolated and purified.

Synthesis of hydroxyalkoxyalkyl dPGs As illustrated in Reaction Scheme 14, Step 2, hydroxyalkyl (119) and/or (120) is/are reacted with an ω-halo-alkan-1-ol (121) in the presence of NaH. The alkanol (121) is added slowly, dropwise, to minimize the formation of dimers. The reaction mixture is allowed to stir for about 24 hours at about 60° C. to afford one or all of the corresponding hydroxyalkyl/hydroxyalkoxy products (122), (123) and/or (124), which can be carried forward together or conventionally separated (e.g., by gel permeation chromatography or size exclusion chromatography) and carried forward independently. The products can be conventionally isolated and purified.

Synthesis of dPGC where R is alkoxy-sulfate or alkoxy-alkyl-sulfate As illustrated in Reaction Scheme 14, Step 3, dPG (122), (123) and/or (124) is/are sulfated, for example, by reaction with pyridine sulfur trioxide complex; the reaction takes place in a suitable solvent (e.g., dry DMF) at about 60° C. for about 12 hours. The reaction is then quenched with water, and the pH is adjusted to 8 (e.g., by addition of NaOH solution). The solvent is evaporated (e.g., under reduced pressure) and the product(s) is/are dissolved (e.g., in brine). Dialysis is performed with a NaCl solution, using an ever-decreasing NaCl concentration, until the medium is changed with distilled water. The final product(s) (Formula Ir, Formula Is and/or Formula It) is/are obtained, e.g., after lyophilization, conventionally separated (e.g., by gel permeation chromatography or size exclusion chromatography) if necessary, conventionally isolated and purified.

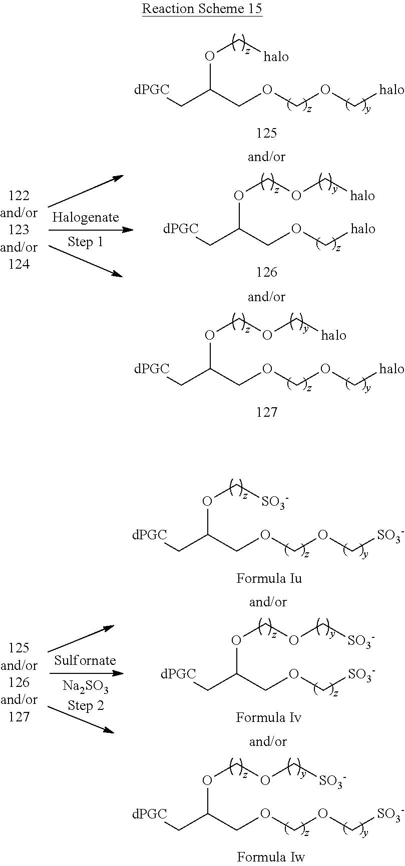

Synthesis of haloalkyl and haloalkoxy dPGs As illustrated in Reaction Scheme 15, Step 1, an hydroxyalkyl/hydroxyalkoxy dPG (122), (123) and/or (124), prepared, e.g., as described with regard to Reaction Scheme 14, is halogenated (e.g., brominated, as described in Zhou et al., Polym. Chem., 2017, 8, 2189) with stirring in a suitable solvent (e.g., anhydrous CH₂Cl₂) using an excess (e.g., 5 eq. to —OH) of tetrabutylammonium bromide (TBAB). To the stirring solution is added a similar excess of 1,8-diazabicyclo(5.4.0)undec-7-ene (DBU) followed by a similar excess of XtalFluor-E (Aldrich), cooling to 0° C. Stirring is continued at room temperature for about 24 hours. The mixture is precipitated (e.g., into methanol/water) with several drops of hydrochloric acid to afford one or all of the corresponding halogenated products (125), (126) and/or (127) where “halo” is bromo, chloro, fluoro or iodo, particularly bromo, which can be carried forward together or conventionally separated (e.g., by gel permeation chromatography or size exclusion chromatography) and carried forward independently. The products can be conventionally isolated and purified.

Synthesis of dPGC where R is alkyl-sulfonate or alkoxy-alkyl-sulfonate As illustrated in Reaction Scheme 15, Step 2, a haloalkyl or haloalkoxy dPG of formulae (125), (126) and/or (127) is contacted with sodium sulfite (about 2 eq.) in a suitable solvent such as methanol and DI water. The mixture is refluxed (at about 102° C.) for about 48 hours to afford the corresponding sulfonated dPG products of Formula Iu, Formula Iv and/or Formula Iw, which can be conventionally separated (e.g., by gel permeation chromatography or size exclusion chromatography) if necessary, and conventionally isolated and purified (e.g., diethyl ether extraction, evaporation, drying and removal of inorganic salts with pure ethanol and filtration).

### Particular Processes and Last Steps

A compound of Formula I is prepared by a thiol-ene click reaction between a dPG-allyl and an ω-mercaptoalkyl carboxylic acid.

A compound of Formula I is contacted with a pharmaceutically acceptable acid to form the corresponding acid addition salt.

A pharmaceutically acceptable acid addition salt of Formula I is contacted with a base to form the corresponding free base of Formula I.

A mercapto-alkyl sulfonate is conjugated to a dPG-allyl functionalized core in a thiol-ene click reaction under UV light to give the corresponding mercapto alkyl sulfonate functionalized dPG.

Both hydroxyl groups of a dPGC-alkanol are sulfated with pyridine sulfur trioxide complex to give the corresponding dPGC-alkanol sulfate/sulfate.

A dPGC-alkylthioalkanol/alkylthioalkyl is sulfated with pyridine sulfur trioxide complex to give the corresponding dPGC-alkyl-thio-alkyl sulfonate/alkylthioalkyl.

### Particular Compounds

The compounds, pharmaceutical formulations, methods of manufacture and use of the present disclosure include the following combinations and permutations of substituent groups of Formula I (sub-grouped, respectively, in increasing order of specificity):
R contributing to DF is selected from the group comprising: -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH.
   R contributing to DF is selected from the group comprising: -(optionally substituted C₈ to C₁₅ alkyl)-COOH, —(C₈ to C₁₅ alkene)-COOH, —(CH₂)_{z}—O—(CH₂)_{y}—COOH, and —(CH₂)_{z}—S—(CH₂)_{y}—COOH.
      y is from about 8 to 13, z is from about 2 to 5, and y+z is from about 10 to 16.
      R not contributing to DF is selected from the group comprising —H, —COOH and —CH₂—CH═CH₂.
         y is from about 8 to 13, z is from about 2 to 5, and y+z is from about 10 to 16.
         dPGC has an average molecular weight of 10 kDa.
         COOH is COO⁻Na⁺.
         DF is at least about 50%.
         DF is at least about 85-95%.
         y is from about 9 to 11 and z is 3.
         dPGC has an average molecular weight of 10 kDa.
         COOH is COO⁻Na⁺.
         DF is at least about 50%.
         DF is at least about 85-95%.
         y is from about 9 to 11 and z is 3.
         dPGC has an average molecular weight of 10 kDa.
         COOH is COO⁻Na⁺.
         DF is at least about 50%.
         DF is at least about 85-95%.
   R contributing to DF is selected from the group comprising: -(optionally substituted C₈ to C₁₅ alkyl)-COOH, —(CH₂)_{z}—O—(CH₂)_{y}—COOH, and —(CH₂)_{z}—S—(CH₂)_{y}—COOH.
      y is from about 8 to 13, z is from about 2 to 5, and y+z is from about 10 to 16.
         y is from about 9 to 11 and z is 3.
      R not contributing to DF is selected from the group comprising —H, —COOH and —CH₂—CH═CH₂.
         y is from about 8 to 13, z is from about 2 to 5, and y+z is from about 10 to 16.
         dPGC has an average molecular weight of 10 kDa.
         COOH is COO⁻Na⁺.
         DF is at least about 50%.
         DF is at least about 85-95%.

Particularly among the compounds, pharmaceutical formulations, methods of manufacture and use of the present disclosure are the following:
dPG_{10 kDa}-C₃SC₁₀—COO⁻Na⁺_{100%},
dPG_{10 kDa}-C₃SC₁₀—COO⁻Na⁺_{95%}/allyl_{5%}, and
dPG_{10 kDa}-C₃SC₁₀—COO⁻Na⁺_{70%}/allyl_{30%}.

The compounds, pharmaceutical formulations, methods of manufacture and use of the present disclosure include the following (sub-grouped, respectively, in increasing order of specificity):
dPG_{10 kDa}-C₃SC₁₀—COO⁻Na⁺_{100%}.
   The dPGC size is about 5 nm and average molecular weight is about 10 kDa
   DF is at least about 40%
      DF is at least 50%
         DF is about 50%.
         R contributing to DF is selected from the group comprising: -(optionally substituted C₅ to C₃₀ alkyl)-SO₃H, -(optionally substituted C₅ to C₃₀ alkyl)-OSO₃H, —(CH₂)_{z}—O—(CH₂)_{y}—SO₃H, —(CH₂)_{z}—O—(CH₂)_{y}—OSO₃H, —(CH₂)_{z}—S—(CH₂)_{y}—SO₃H, and —(CH₂)_{z}—S—(CH₂)_{y}—OSO₃H.
         R contributing to DF is selected from the group comprising: —(C₈ to C₁₅ alkyl)-SO₃H, —(C₈ to C₁₅ alkyl)-OSO₃H, —(CH₂)_{z}—S—(CH₂)_{y}—SO₃H, and —(CH₂)_{z}—S—(CH₂)_{y}—OSO₃H.
         R not contributing to DF is selected from the group comprising: —C₈ to C₁₅ alkenyl, C₈ to C₁₅ ω-hydroxyalkyl, and C₈ to C₁₅ ω-hydroxyalkylthioalkyl.
         R not contributing to DF is H, SO₃, —CH₂—CH═CH₂ or —(CH₂)_{z}—S—(CH₂)_{y}—CH₃.
         y is from about 8 to 13, z is from about 2 to 5, and y+z is from about 10 to 16.
         y is from about 8 to 13, z is from about 2 to 5, and y+z is from about 10 to 16.
         R not contributing to DF is H, SO₃, —CH₂—CH═CH₂ or —(CH₂)_{z}—S—(CH₂)_{y}—CH₃.
         y is from about 8 to 13, z is from about 2 to 5, and y+z is from about 10 to 16.
         R not contributing to DF is selected from the group comprising: —C₈ to C₁₅ alkenyl, C₈ to C₁₅ ω-hydroxyalkyl, and C₈ to C₁₅ ω-hydroxyalkylthioalkyl.
         R not contributing to DF is H, SO₃, —CH₂—CH═CH₂ or —(CH₂)_{z}—S—(CH₂)_{y}—CH₃.
         y is from about 8 to 13, z is from about 2 to 5, and y+z is from about 10 to 16.
         R contributing to DF is selected from the group comprising: —(C₈ to C₁₅ alkyl)-SO₃H, —(C₈ to C₁₅ alkyl)-OSO₃H, —(CH₂)_{z}—S—(CH₂)_{y}—SO₃H and —(CH₂)_{z}—S—(CH₂)_{y}—OSO₃H.
         R not contributing to DF is selected from the group comprising: —C₈ to C₁₅ alkenyl, C₈ to C₁₅ ω-hydroxyalkyl, and C₈ to C₁₅ ω-hydroxyalkylthioalkyl.
         R not contributing to DF is H, SO₃, —CH₂—CH═CH₂ or —(CH₂)_{z}—S—(CH₂)_{y}—CH₃.
         y is from about 8 to 13, z is from about 2 to 5, and y+z is from about 10 to 16.
R contributing to DF is selected from the group comprising: -(optionally substituted C₅ to C₃₀ alkyl)-SO₃H, -(optionally substituted C₅ to C₃₀ alkyl)-OSO₃H, —(CH₂)_{z}—O—(CH₂)_{y}—SO₃H, —(CH₂)_{z}—O—(CH₂)_{y}—OSO₃H, —(CH₂)_{z}—S—(CH₂)_{y}—SO₃H, and —(CH₂)_{z}—S—(CH₂)_{y}—OSO₃H.
   DF is at least about 40%
   DF is at least 50%
      DF is about 50%.
   R contributing to DF is selected from the group comprising:
      —(C₈ to C₁₅ alkyl)-SO₃H, —(C₈ to C₁₅ alkyl)-OSO₃H, —(CH₂)_{z}—S—(CH₂)_{y}—SO₃H and —(CH₂)_{z}—S—(CH₂)_{y}—OSO₃H.
         R not contributing to DF is selected from the group comprising: —C₈ to C₁₅ alkenyl, C₈ to C₁₅ ω-hydroxyalkyl, and C₈ to C₁₅ ω-hydroxyalkylthioalkyl.
         R not contributing to DF is H, SO₃, —CH₂—CH═CH₂ or —(CH₂)_{z}—S—(CH₂)_{y}—CH₃.
         y is from about 8 to 13, z is from about 2 to 5, and y+z is from about 10 to 16.
         DF is at least 40%
         DF is at least 50%
         DF is about 50%.
         y is from about 8 to 13, z is from about 2 to 5, and y+z is from about 10 to 16.
         R not contributing to DF is H, SO₃, —CH₂—CH═CH₂, or —(CH₂)_{z}—S—(CH₂)_{y}—CH₃.
         y is from about 8 to 13, z is from about 2 to 5, and y+z is from about 10 to 16.
   R not contributing to DF is selected from the group comprising:
   —C₈ to C₁₅ alkenyl, C₈ to C₁₅ ω-hydroxyalkyl, and C₈ to C₁₅ ω-hydroxyalkylthioalkyl.
   R not contributing to DF is H, SO₃, —CH₂—CH═CH₂, or —(CH₂)_{z}—S—(CH₂)_{y}—CH₃.
   y is from about 8 to 13, z is from about 2 to 5, and y+z is from about 10 to 16.
R contributing to DF is selected from the group comprising: —(C₈ to C₁₅ alkyl)-SO₃H, —(C₈ to C₁₅ alkyl)-OSO₃H, —(CH₂)_{z}—S—(CH₂)_{y}—SO₃H, and —(CH₂)_{z}—S—(CH₂)_{y}—OSO₃H.
R not contributing to DF is selected from the group comprising: —C₈ to C₁₅ alkenyl, C₈ to C₁₅ ω-hydroxyalkyl, and C₈ to C₁₅ ω-hydroxyalkylthioalkyl.
R not contributing to DF is H, SO₃, —CH₂—CH═CH₂, or —(CH₂)_{z}—S—(CH₂)_{y}—CH₃.
y is from about 8 to 13, z is from about 2 to 5, and y+z is from about 10 to 16.
DF is at least 40%, at least 50%, about 40 to 70%, or about 50%

Alternatively, for the compounds, pharmaceutical formulations, methods of manufacture and use of the present disclosure dPGC has a size from about 4 to 10 nm and an average molecular weight from about 5 to 25 kDa.

### Utility, Testing, Administration and Formulation

### General Utility

The compositions of the disclosure find use in a variety of applications. As will be appreciated by those in the art, the compositions are antiviral and have demonstrated virucidal activity against HSV-2 and surprisingly against SARS-CoV-2. The compounds of the disclosuree are useful for treating an HSPG dependent virus, i.e., a virus that exploits heparan sulfate proteoglycans (HSPGs) as attachment receptors on eukaryotic cells. In certain embodiments, the HSPG dependent virus is selected from the group comprising HIV-1, HSV (HSV-1, HSV-2), HCMV, HPV, Respiratory syncytial virus (RSV) and filoviruses. In other embodiments of the disclosure, the virus is selected from the group comprising HIV-1, HSV (HSV-1, HSV-2), HCMV, HPV, Respiratory syncytial virus (RSV), influenza virus, and filoviruses. Diseases associated with such viruses are selected from the group comprising respiratory viral diseases, gastrointestinal viral diseases, exanthematous viral disease, hepatic viral diseases, cutaneous viral diseases, hemorrhagic viral diseases, neurologic viral diseases. Typical diseases are, but not limited to, the common cold, flu, warts, viral herpes, hepatitis, rubella, smallpox, HIV/AIDS, Ebola, SARS-CoV-2/COVID-19, polio, viral meningitis, etc. . . . .

An aspect of the disclosure provides a method of treating and/or preventing COVID-19 and other respiratory diseases caused by coronaviruses, influenza virus infections and/or diseases associated therewith, comprising administering to a subject in need thereof, a therapeutically effective amount of one or more virucidal compositions of the disclosure. These treatable viruses and diseases are discussed in greater detail below.

Coronaviruses (CoVs) are abundant and tend to cause mild to serious upper-respiratory tract syndromes, like the common cold or lower respiratory diseases like wheezy bronchitis and other affections of the lower respiratory tract. Coronaviruses tend to reinfect the same human hosts (Archives of Disease in Childhood, 1983, 58, 500-503). Coronaviruses are zoonotic and circulate among pigs, horses, cats, bats, camels, among other species. When a coronavirus jumps from an animal to humans, they can cause the mild to moderate diseases associated to coronaviruses such as HCV229E (alpha CoV), HCVOC43 (beta CoV), HCVNL63 (alpha CoV), HCVOC43 (beta coronavirus), HCVHKU1 (beta coronavirus), all of which do not have a distinct pathognomonic syndrome named after the individual virus. In the last two decades, three CoVs have caused serious respiratory (upper and lower) syndromes with dedicated syndromes being named to describe their infections: MERS-CoV (beta CoV that causes Middle East Respiratory Syndrome, or MERS); SARS-CoV (the beta CoV that causes severe acute respiratory syndrome, or SARS) and SARS-CoV-2 (the novel CoV that causes COVID-19). Like all viruses, CoVs use either Sialic Acids (SAs) and/or Heparan Sulfate Proteoglycans (HSPGs), among other cell surface receptors such as the Angiotensin Converting Enzyme to infect the host cell. HCVNL63 and SARS-CoV use primarily SAs to dock onto host cells while the docking used by other variants is still under investigation (Microorganisms 2020, 8, 1894; doi:10.3390/microorganisms8121894).

CoVs are also of great importance in the veterinary and livestock industries because they cause diseases to animals. The Equine Coronavirus (ECoV), a beta-CoV causes enteric inflammation on horses and is closely related to the bovine CoV (BCoV), also a beta-CoV that causes enzootic pneumonia complex and dysentery in calves and has been reported to cause winter dysentery in adult cattle. Both ECoV and BCoV infect the host cells via the N-acetyl-9-O-acetylneuraminic acid receptor, also referred to as Sialic acid. In pigs, the Porcine Respiratory Coronavirus (PRCv) causes a respiratory disease to which the only treatment is isolation of the contaminated animal. Other CoVs affect pigs, such as Transmissible Gastroenteritis Virus (TGEV), Porcine epidemic diarrhoea virus (PEDV), and porcine haemagglutinating encephalomyelitis virus (PHEV). PDCoV (porcine deltacoronavirus) TGEV and PRCV are alpha CoVs and closely associated to the CoVs that affect cats and dogs, and to PEDV and human CoVs HCV229E and HCVNL63. PHEV and PDCoV are the beta CoVs. Poultry and many avian species also develop diseases caused by CoVs such as coronaviruses of the domestic fowl—infectious bronchitis virus IBV, that causes respiratory illness to chicken (Gallus gallus), turkey (Meleagris gallopavo) and pheasant (Phasianus colchicus). Improvements in testing and detection will likely increase the list of coronaviruses that affect animals. The fear of new outbreaks of CoVs relevant to human health may also increase this list as the source of new outbreaks lies predominantly in livestock.

Certain compositions and methods provided herein are particularly deemed useful for the treatment of COVID-19.

Another aspect of the disclosure provides a method of disinfection and/or sterilization of surfaces using one or more compounds of the disclosure or the virucidal composition of the disclosure or the pharmaceutical composition of the disclosure. The disinfection and/or sterilization is done on living surfaces or non-living surfaces. The living surfaces are human or animal skin and/or hair. The non-living surface are, but not limited to, medical equipment, touch screens, textile, clothing, masks, gloves, furniture, and any other surfaces present in rooms, transport means, public spaces such as schools, airports, public transportation and cinemas. In some other embodiments, the non-living surfaces are fabric surfaces (masks, gloves, doctor coats, curtains, bed sheet), metal surfaces (lifts, door handle, nobs, railings, medical equipment and instruments, public transport and places), wood material surfaces (furniture, floors, partition panels), concrete surfaces (hospitals, clinics and isolation wards and walls), and plastic surfaces (medical equipment and instruments, touch screens, switches, kitchen and home appliances).

In an embodiment, the method of disinfection and/or sterilization of surfaces comprises the steps of (i) providing at least one compound of the disclosure or a virucidal composition of the disclosure, or pharmaceutical composition of the disclosure, (ii) contacting a virus-contaminated surface or a surface suspected to be contaminated by a virus with the at least one compound of the disclosure or a virucidal composition of the disclosure or pharmaceutical composition of the disclosure for a time sufficient to obtain virucidal effect. In some embodiments, the virus-contaminated surface is human or animal skin and/or hair. In other embodiments, the virus-contaminated surface is a non-living surface. The non-living surface is, but not limited to, medical equipment, touch screens, textile, clothing, masks, gloves, furniture, and any other surfaces present in rooms, transport means, public spaces such as schools, airports, public transportation and cinemas. In some other embodiments, the non-living surfaces are fabric surfaces (masks, gloves, doctor coats, curtains, bed sheet), metal surfaces (lifts, door handle, nobs, railings, medical equipment and instruments, public transport and places), wood material surfaces (furniture, floors, partition panels), concrete surfaces (hospitals, clinics and isolation wards and walls), and plastic surfaces (medical equipment and instruments, touch screens, switches, kitchen and home appliances).

Another aspect of the disclosure provides a use of a compound of the disclosure or a virucidal composition of the disclosure or a pharmaceutical composition of the disclosure for sterilization and/or for disinfection. In some embodiments, sterilization and disinfection is for virus-contaminated surfaces or surfaces suspected to be contaminated by a virus. In some embodiments, the surfaces are human or animal skin and/or hair. Thus in some embodiments, the disclosure provides a use of a compound of the disclosure or a virucidal composition of the disclosure or a pharmaceutical composition of the disclosure for sterilization and/or for disinfection of human or animal skin and/or hair. In other embodiments, the surfaces are non-living surfaces. The non-living surfaces are, but not limited to, medical equipment, touch screens, textile, clothing, masks, gloves, furniture, and any other surfaces present in rooms, transport means, public spaces such as schools, airports, public transportation and cinemas. In some other embodiments, the non-living surfaces are fabric surfaces (masks, gloves, doctor coats, curtains, bed sheet), metal surfaces (lifts, door handle, nobs, railings, medical equipment and instruments, public transport and places), wood material surfaces (furniture, floors, partition panels), concrete surfaces (hospitals, clinics and isolation wards and walls), and plastic surfaces (medical equipment and instruments, touch screens, switches, kitchen and home appliances). In an embodiment, the virucidal composition of the disclosure or the pharmaceutical composition of the disclosure is used as virucidal hand disinfectant for frequent use. In another embodiment, the virucidal composition of the disclosure or the pharmaceutical composition of the disclosure is applied by spraying. In a further embodiment, the virucidal composition of the disclosure of the pharmaceutical composition of the disclosure is applied on a protective mask.

Another aspect of the disclosure provides a use of the compounds of the disclosure or the virucidal composition of the disclosure for manufacturing (producing) virucidal surfaces (i.e. able to inactivate viruses). Such surfaces are, but not limited to, textile, clothing, masks, touch screens, medical equipment, furniture. In some other embodiments, the surfaces are fabric surfaces (masks, gloves, doctor coats, curtains, bed sheet), metal surfaces (lifts, door handle, nobs, railings, medical equipment and instruments, public transport and places), wood material surfaces (furniture, floors, partition panels), concrete surfaces (hospitals, clinics and isolation wards and walls), and plastic surfaces (medical equipment and instruments, touch screens, switches, kitchen and home appliances). In some embodiments, the surfaces can be modified with the one or more compounds of the disclosure either through chemical modification or physical coating known in the art. Examples of physical coating are spraying or dipping the surface in a solution comprising the one or more compounds of the disclosure.

Another aspect of the disclosure provides a method for manufacturing (producing) a virucidal surface, wherein the method comprises coating the surface with the one or more compounds of the disclosure or the virucidal composition of the disclosure. The surface is, but not limited to, textile, clothing, masks, touch screens, medical equipment, furniture. In some other embodiments, the surface is fabric surface (masks, gloves, doctor coats, curtains, bed sheet), metal surface (lifts, door handle, nobs, railings, medical equipment and instruments, public transport and places), wood material surface (furniture, floors, partition panels), concrete surface (hospitals, clinics and isolation wards and walls), and plastic surface (medical equipment and instruments, touch screens, switches, kitchen and home appliances). The coating can be done either through chemical modification or physical coating known in the art.

Another aspect of the disclosure provides a virucidal surface coating composition comprising the one or more compounds of the disclosure or the virucidal composition of the disclosure. The virucidal surface coating composition of the disclosure can be sprayed or painted on surfaces. The surfaces are, but not limited to, medical equipment, touch screens, textile, clothing, masks, gloves, furniture, and any other surfaces present in rooms, transport means, public spaces such as schools, airports, public transportation and cinemas. In some other embodiments, the surfaces are fabric surfaces (masks, gloves, doctor coats, curtains, bed sheet), metal surfaces (lifts, door handle, nobs, railings, medical equipment and instruments, public transport and places), wood material surfaces (furniture, floors, partition panels), concrete surfaces (hospitals, clinics and isolation wards and walls), and plastic surfaces (medical equipment and instruments, touch screens, switches, kitchen and home appliances).

Another aspect of the disclosure provides a device comprising a surface coated with one or more compounds of the disclosure or with the virucidal composition of the disclosure. Such an antiviral coated device can be, but is not limited to, clothing, a mask, a glove, a touch screen, medical equipment, furniture, etc. . . . . In one embodiment, the device is a mask, clothing or medical equipment. In another embodiment, the device is a medical device.

### Testing

The weight, size, and degree of functionalization of the compounds of the disclosure can be determined, for example, by gel permeation chromatography, by dynamic light scattering and by nuclear magnetic resonance, as described in Bhatia, et al., “Linear polysialoside outperforms dendritic analogs for inhibition of influenza virus infection in vitro and in vivo”, Biomaterials, Vol. 138, 22-34 (September 2017) ((DOI: 10.1039/c7py01470h) (https://doi.org/10.1016/j.biomaterials.2017.05.028)

Degree of branching can be calculated using inverse gated (IG)¹³C NMR, for example, as described in Haag, R.; Sunder, A.; Stumbe, J.-F. J Am Chem Soc 2000, 122, 2954.

In vitro activity for SARS-CoV-2 inhibition is determined, for example, as described in Gasbarri et al., Microorganisms 2020, 8, 1894 (2020).

In vivo activity for SARS-CoV-2 inhibition is determined, for example, as described in Kaptein et al., “Favipiravir at high doses has potent antiviral activity in SARS-CoV-2-infected hamsters, whereas hydroxychloroquine lacks activity,” Proc Natl Acad Sci USA, 2020 Oct. 27; 117(43): 26955-26965, or in Imai et al., “Syrian hamsters as a small animal model for SARS-CoV-2 infection and countermeasure development,” https://www.pnas.org/content/117/28/16587, or in Rosenke et al., “Orally delivered MK-4482 inhibits SARS-CoV-2 replication in the Syrian hamster model,” https://www.nature.com/articles/s41467-021-22580-8.

Cytotoxicity is determined by exposing Vero cells to varying concentrations of test drug and measuring the percentage of cells surviving such exposure. Antiviral activity is determined by plaque reduction assays on infected Vero cells, measuring the number of plaques that form in wells exposed to a mixture of a fixed concentration of the virus and varying concentrations of test drug. Virucidal activity is determined by exposing Vero cells to different dilutions of a prep-incubated mixture of virus and an effective amount of test drug. After incubation, the solution is removed and the cells are incubated again, measuring the plaques that form, evaluating the viral titer. The decrease of viral titer with respect to an untreated control is an indication of virucidal activity. These determinations can be carried out, for example, as described in Cagno, et al., “Broad-spectrum non-toxic antiviral nanoparticles with a virucidal inhibition mechanism,” Nature Materials 17, 195-203 (2018).

### Administration

The compounds of Formula I are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities.

While human dosage levels have yet to be optimized for the compounds of the disclosure, generally, a daily dose is from about 0.001 to 2.0 mg/kg of body weight/day, particularly about 0.005 to 0.75 mg/kg of body weight/day, and most particularly about 0.01 to 0.5 mg/kg of body weight. Thus, for administration to a 70 kg person, the dosage range would be about 0.07 to 140 mg per day, particularly about 0.35 to 52.5 mg per day, and most particularly about 0.7 to 35 mg per day. Administration can be as a single daily dose or divided into 2 or more doses per day, over a period of treatment lasting from about 1 to about 7 days. The amount of active compound administered will, of course, be dependent on the subject and disease state being treated, the severity of the affliction, the manner and schedule of administration and the judgment of the prescribing physician.

### Formulation

The compounds of the disclosure that are used in the methods of the present disclosure can be incorporated into a variety of formulations and medicaments for therapeutic administration. More particularly, the compounds as provided herein can be formulated into pharmaceutical compositions by combination with appropriate, pharmaceutically acceptable carriers, excipients and/or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, inhalation (pulmonary, nasal), rectal, parenteral, intraperitoneal, intradermal, topical, transdermal, intracranial and/or intratracheal administration. Moreover, the compounds can be administered in a local rather than systemic manner, e.g., in a topical cream or gel, a depot or a sustained release formulation. The compounds can be formulated with common excipients, diluents or carriers, and compressed into tablets, or formulated as elixirs or solutions for convenient oral administration, or administered by the intramuscular or intravenous routes. The compounds can be administered alone, in combination with each other, or they can be used in combination with other known compounds including other antiviral agents. Suitable formulations for use in the present disclosure are found in Remington's Pharmaceutical Sciences (Mack Publishing Company (1985) Philadelphia, Pa., 17th ed.), which is incorporated herein by reference. Moreover, for a brief review of methods for drug delivery, see, Langer, Science (1990) 249:1527-1533, which is incorporated herein by reference.

As to the appropriate excipients, carriers and diluents, reference may be made to the standard literature describing these, e.g. to chapter 25.2 of Vol. 5 of “Comprehensive Medicinal Chemistry”, Pergamon Press 1990, and to “Lexikon der Hilfsstoffe für Pharmazie, Kosmetik und angrenzende Gebiete”, by H. P. Fiedler, Editio Cantor, 2002. The term “pharmaceutically acceptable carrier, excipient and/or diluent” means a carrier, excipient or diluent that is useful in preparing a pharmaceutical composition that is generally safe, and possesses acceptable toxicities. Acceptable carriers, excipients or diluents include those that are acceptable for veterinary use as well as human pharmaceutical use. A “pharmaceutically acceptable carrier, excipient and/or diluent” as used in the specification and claims includes both one and more than one such carrier, excipient and/or diluent.

Sustained-release preparations can be prepared. Suitable examples of sustained-release preparations include semi-permeable matrices of solid hydrophobic polymers containing the compounds of the disclosure, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma] ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The compounds of the disclosure can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting. For injection, a compound of the disclosure (and optionally another active agent) can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. Particularly, the compounds of the disclosure can be formulated in aqueous solutions, particularly in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Pharmaceutical formulations for parenteral administration include aqueous solutions of the compounds of the disclosure in water-soluble form. Additionally, suspensions of the compounds of the disclosure can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds of the disclosure to allow for the preparation of highly concentrated solutions.

The amount of a compound of the disclosure that can be combined with a carrier material to produce a single dosage form will vary depending upon the viral disease treated, the mammalian species, and the particular mode of administration. It will be also understood, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular viral disease undergoing therapy, as is well understood by those of skill in the area.

Another aspect of the disclosure provides a virucidal composition comprising an effective amount of one or more compounds of the disclosure and optionally at least one suitable carrier or aerosol carrier. “An effective amount” refers to the amount sufficient for irreversibly inhibiting viruses; i.e. sufficient for obtaining virucidal effect. In an embodiment, the suitable carrier is selected from the group comprising stabilisers, fragrance, colorants, emulsifiers, thickeners, wetting agents, or mixtures thereof. In another embodiment, the virucidal composition can be in the form of a liquid, a gel, a foam, a spray or an emulsion. In a further embodiment, the virucidal composition can be an air freshener, a sterilizing solution or a disinfecting solution.

Another aspect of the disclosure provides a device (or a product) comprising the virucidal composition of the disclosure or one or more compounds of the disclosure and means for applying and/or dispensing thereof (i.e. the compounds of the disclosure or the virucidal composition). In another embodiment, the means comprise a dispenser, a spray applicator or a solid support soaked with the compounds of the disclosure. In another embodiment, the support is a woven or non-woven fabric, a textile, a paper towel, cotton wool, an absorbent polymer sheet, or a sponge.

Nasal solutions of the active compound alone or in combination with other pharmaceutically acceptable excipients can also be administered.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, particularly less than 10 microns.

Those skilled in the art will appreciate that the disclosure described herein is susceptible to variations and modifications other than those specifically described. It is to be understood that the disclosure includes all such variations and modifications without departing from the spirit or essential characteristics thereof. The disclosure also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the disclosure being indicated by the appended claims, and all changes which come within the meaning and range of equivalency are intended to be embraced therein.

### EXAMPLES

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes. All references cited herein are incorporated by reference in their entirety.

### Example 1

### dPG-Carboxylate

1A. Preparation of dPG_{10 kDa} (3): Trimethylolpropane (TMP) (25 mmol) was deprotonated by potassium methoxide solution (10 mmol KOH in 10 mL methanol). The resulting methanol was evaporated at 60° C. under vacuum (3 mbar). The synthesis-reactor was then heated to 100° C. and glycidol was added slowly over a period of 24 h. The resulting dPG, having an average molecular weight of 10 kDa (“dPG_{10 kDa}” or “C1”), was used for functionalization. The product was characterized by NMR and GPC (H₂O) for the determination of absolute molecular weights and polydispersity index [GPC (H₂O): Mₙ=9.6 kDa, M_{w}=12.6 kDa, D=1.31]. The degree of branching (DB) was calculated to be 59%, using inverse gated (IG)¹³C NMR, as reported in literature.

By varying the amount of glycidol and adjusting the period of addition, the corresponding dPGs, having average molecular weights of 5 kDa (“dPG_{5 kDa}”), 25 kDa (“dPG_{25 kDa}”), and 100 kDa (“dPG_{100 kDa}”) were similarly obtained.

1B. Preparation of dPG_{10 kDa}-allyl (5): Using the product obtained, e.g., as described in Example 1A, dPG_{10 kDa} (200 mg, 2.69 mmol OH to be functionalized) was dried at 60° C. overnight under high vacuum. The dried dPG was dissolved in dry DMF (20 mL) and cooled to 0° C. in an ice bath. To the stirred solution of dPG in dry DMF at 0° C., NaH (129.12 mg, 5.38 mmol, 2 eq.) was added. After the NaH addition, the ice bath was removed and the temperature of the reaction mixture was allowed to reach room temperature. The reaction mixture was allowed to stir for 1 hour at room temperature, then stirred for 1 hour at 40° C. and then cooled down again using an ice bath. The allyl bromide (3-bromoprop-1-ene) (465 μL, 5.38 mmol, 2.0 eq.) in dry DMF (1 mL) was added dropwise to the reaction mixture using a syringe. The ice bath was removed and after stirring for 24 hours at 40° C. the reaction was quenched by addition of methanol and the resulting mixture was dialyzed in MeOH to afford dPG-allyl (DF=100). Degree of allylation was quantified by ¹H NMR in CD₃OD.

1C. Preparation of Formula I: In dPG-allyl (DF=100) (50 mg, 0.67 mmol of allyl group) and 11-mercaptoundecanoic acid (1.35 mmol, 295 mg) were dissolved in methanol (5 mL). 2,2-Dimethoxy-2-phenylacetophenone (DMPA) as radical initiator (50 mg, 0.19 mmol) and a catalytic amount of tris(2-carboxyethyl)phosphine hydrochloride (TCEP-HCl) were added to the reaction to avoid oxidation of the thiol intermediate. Upon observing precipitation, a few drops of water were added to dissolve the precipitate and obtain a clear solution. The solution was degassed by flushing argon through the reaction mixture for 10 minutes. The reaction mixture was stirred and irradiated with UV light using a high pressure UV lamp at room temperature for 5 hours. The reaction mixture was dialyzed (MWCO 2 kDa) against methanol/water mixture to remove the TCEP.HCl, DMPA and any excess unreacted thiol. The successful formation of product, dPG_{10 kDa}-C₃SC₁₀—COO⁻Na⁺_{100%}, was confirmed by ¹HNMR of pure product by correlating the aliphatic protons of ligands at 1.5-1.00 ppm with the polyglycerol backbone protons at 3.7-3.2 ppm. In addition, elemental analysis was used for the sulfur content measurement confirming the click reaction.

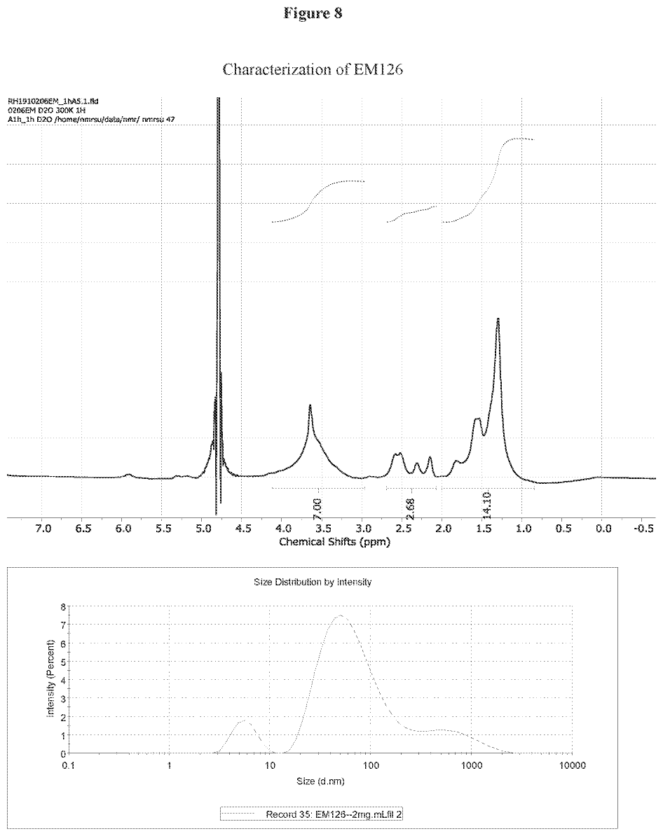

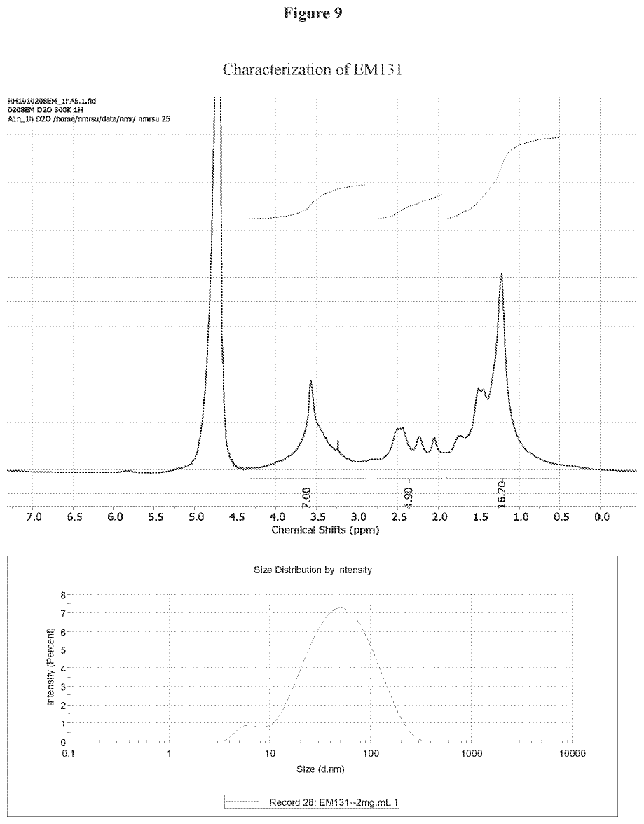

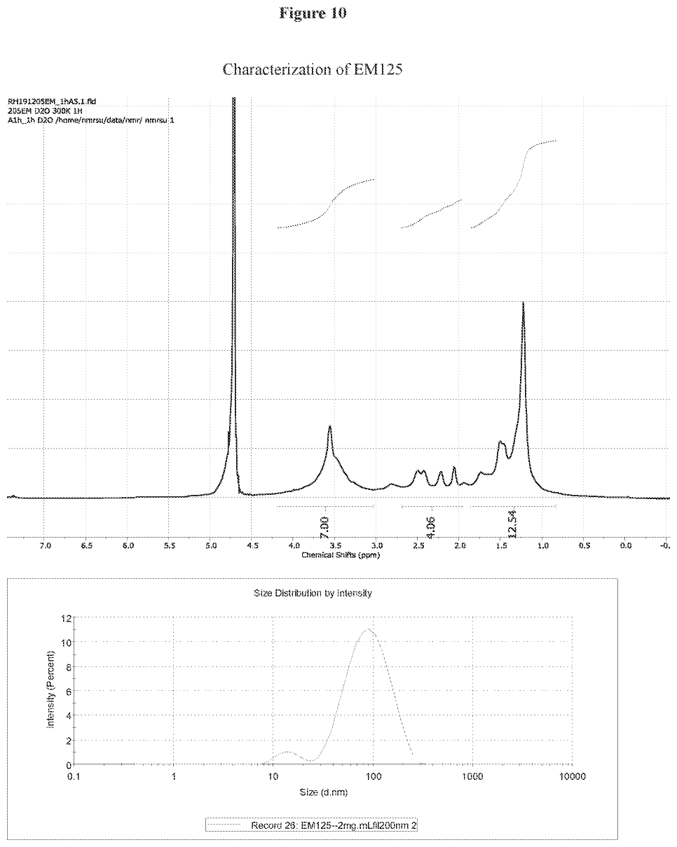

1D. Other Compounds of Formula I: By following the procedures of Examples 1B and 1C and substituting dPG_{10 kDa} with the following compounds as obtained in Example 1A, dPG_{5 kDa}, dPG_{25 kDa} and dPG_{100 kDa}, there were obtained the following:
dPG_{5 kDa}-C₃SC₁₀—COO⁻Na⁺_{100%} (“EM124”),
dPG_{25 kDa}-C₃SC₁₀—COO⁻Na⁺_{100%} (“EM126”) and
dPG_{100 kDa}-C₃SC₁₀—COO⁻Na⁺_{100%} (“EM131”),
   the characterization data for which are shown in FIGS. 7, 8 and 9.

1E. Other Compounds of Formula I: By following the procedures of Examples 1B and 1C and substituting dPG_{10 kDa} with dPG_{5 kDa}, as obtained in Example 1A, and substituting 11-mercaptoundecanoic acid with 12-mercaptododecanoic acid, there was obtained the following:
dPG_{5 kDa}-C₃SC₁₁—COO⁻Na⁺_{100%} (“EM125”),
   the characterization data for which is shown in FIG. 10.

### Example 2

### Other Compounds of Formula I

2A. Formula 5: By following the procedures of Example 1B and substituting allyl bromide with:
a) 4-bromobut-1-ene,
b) 5-bromopent-1-ene,
c) 7-bromo-5-methylhept-1-ene, and
d) 10-iodo-5-isopropyl-6-methyldec-1-ene;
   there are obtained the following compounds, respectively:
a) dPG_{10 kDa}-but-1-ene₁₀₀%,
b) dPG_{10 kDa}-pent-1-ene₁₀₀%,
c) dPG_{10 kDa}-5-methylhept-1-ene_{100%}, and
d) dPG_{10 kDa}-5-isopropyldec-1-ene_{100%}.

2B. Formula I: By following the procedure described in Example 1C and substituting dPG_{10 kDa}-allyl_{100%}s with compounds obtained in Example 2A, there are obtained the following respective compounds:
a) dPG_{10 kDa}-C₄SC₁₀—COO⁻Na⁺_{100%},
b) dPG_{10 kDa}-C₅SC₁₀—COO⁻Na⁺_{100%},
c) dPG_{10 kDa}-C₇(5-Me)SC₁₀—COO⁻Na⁺_{100%}, and
d) dPG_{10 kDa}-C₁₀(5-iPr)SC₁₀—COO⁻Na⁺_{100%}.

2C. Formula I: By following the procedure described in Example 1C and substituting 11-mercaptoundecanoic acid with:
a) 8-mercaptooctanoic acid,
b) 3-(6-merdcaptohexyl)pentanedioic acid,
c) 10-mercaptodecanoic acid,
d) 12-mercaptododecanoic acid, and
e) 13-mercaptotridecanoic acid;
   there are obtained the following compounds, respectively:
a) dPG_{10 kDa}-C₃SC₇—COO⁻Na⁺_{100%},
b) dPG_{10 kDa}-C₃SC₇-(di-CH₂—COO⁻Na⁺)_{100%},
c) dPG_{10 kDa}-C₃SC₉—COO⁻Na⁺_{100%},
d) dPG_{10 kDa}-C₃SC₁₁—COO⁻Na⁺_{100%}, and
e) dPG_{10 kDa}-C₃SC₁₂—COO⁻Na⁺_{100%}.

2D. Formula I: By following the procedures of Example 1B and substituting allyl bromide with:
a) 8-bromooctanoic acid,
b) 9-bromononanoic acid,
c) 10-bromodecanoic, and
d) 11-iodoundecanoic acid;
   there are obtained the following compounds, respectively:
a) dPG_{10 kDa}-C₇—COOH_{100%},
b) dPG_{10 kDa}-C₈—COOH_{100%},
c) dPG_{10 kDa}-C₉—COOH_{100%}, and
d) dPG_{10 kDa}-C₁₀—COOH_{100%}.

2E. Formula 11: By following the procedures of Example 1B and substituting allyl bromide with:
a) 3-bromopropanol, and
b) 4-brombutanol;
   there are obtained the following compounds, respectively:
a) dPG_{10 kDa}-C₃—OH_{100%}, and
b) dPG_{10 kDa}-C₄—OH_{100%}.

2F. Formula I: By following the procedures of Example 1C and substituting dPG_{10 kDa}-allyl_{100%}s with compounds obtained in Example 2E, there are obtained the following respective compounds:
c) dPG_{10 kDa}-C₃OC₁₀—COO⁻Na⁺_{100%}, and
d) dPG_{10 kDa}-C₄OC₁₀—COO⁻Na⁺_{100%}.

### Example 3

### Inhibition of SARS-CoV-2

A. Cells and Virus: Vero C1008 (clone E6) (ATCC CRL-1586) cells are propagated in DMEM High Glucose+Glutamax supplemented with 10% fetal bovine serum (FBS) and 1% penicillin/streptavidin (pen/strep). SARS-CoV2/Switzerland/GE9586/2020 is isolated from a clinical specimen in Vero-E6 and passaged twice before the experiments. Cells are infected with the virus and the supernatant is collected 3 days post infection, clarified, aliquoted, and frozen at −80° C. and subsequently titrated by plaque assay in Vero-E6.

B. Inhibition assay against SARS-CoV-2: The antiviral effect of dendritic polyglycerol against SARS-CoV-2 is tested by plaque reduction assays on Vero-E6 cells. Vero-E6 cells are plated 24 h before the experiment in 24-well plates at a density of 10⁵ cells. A fixed amount of virus (MOI=0.0005) is pre-incubated for 1 hour at 37° C. with serial dilutions of the compound of interest. The solution is then transferred onto the cells and incubated for 1 hour. Afterwards, the solution is removed and the cells incubated for 24 h in DMEM w5% FBS with 0.4 w % Avicel. The cells are then stained with crystal violet and the plaques counted. The inhibition at each concentration is then compared to an untreated control and percentage of inhibition calculated.

C. Virucidal Assay: Viruses (10⁵ pfu of SARS-CoV-2) and test compounds are incubated for 1 h at 37° C., and then the virucidal effect is investigated by adding serial dilutions of the mixtures on Vero-E6 for 1 h, followed by addition of medium containing avicel. Viral titers are determined at dilutions at which the material is not effective.

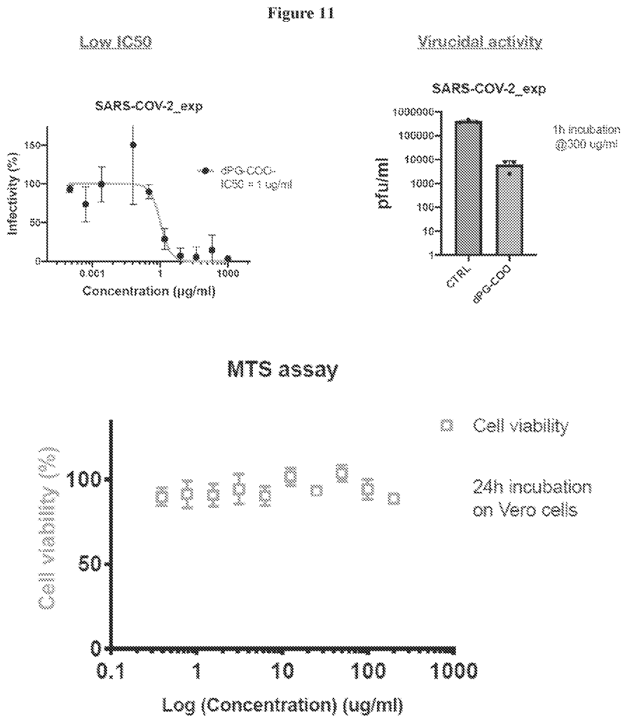

D. Results: When tested as described above in Example 3B, dPG_{10 kDa}-C₃—S—C₁₀—COO⁻Na⁺_{100%} inhibited SarsCoV-2 as shown in FIG. 11. When tested as described above in Example 3C, the composition dPG_{10 kDa}-C₃—S—C₁₀—COO⁻Na⁺_{100%} shows virucidal efficacy as shown in FIG. 11.

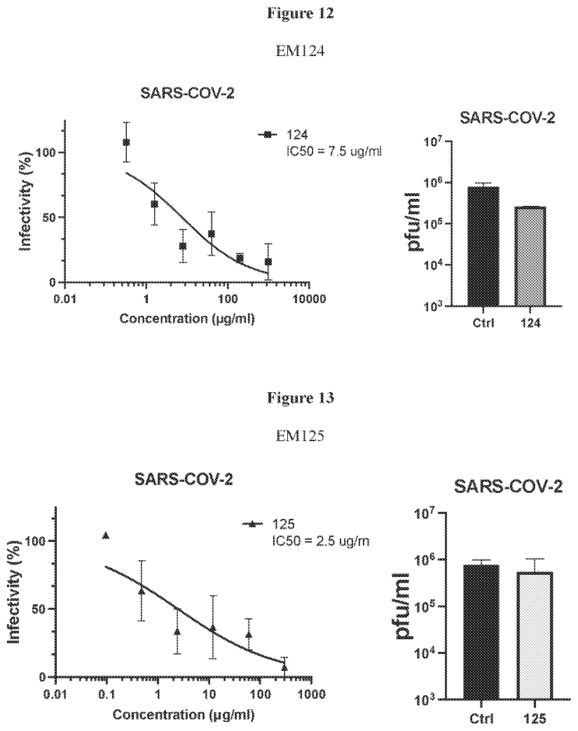

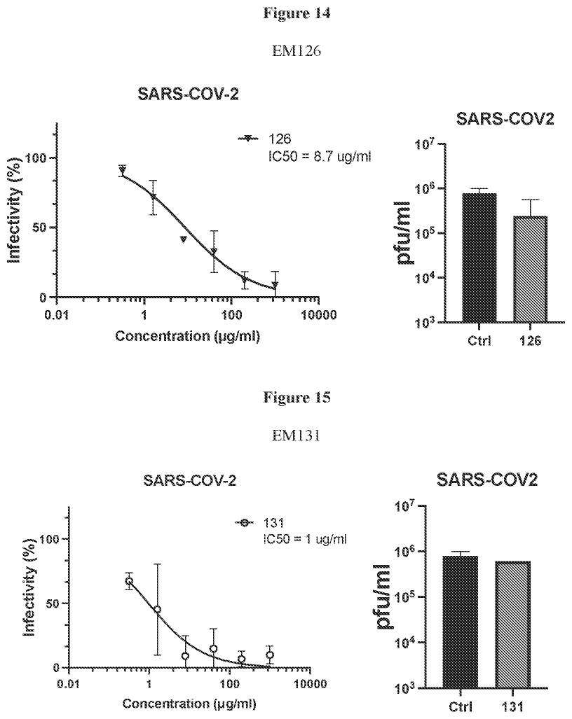

D. Results: When tested as described above in Example 3B and Example 3C, the compounds dPG_{5 kDa}-C₃SC₁₀—COO⁻Na⁺_{100%} (“EM124”), dPG_{5 kDa}-C₃SC₁₁—COO⁻Na⁺_{100%}r (“EM125”), dPG_{25 kDa}-C₃SC₁₀—COO⁻Na⁺_{100%} (“EM126”) and dPG_{100 kDa}-C₃SC₁₀—COO⁻Na⁺_{100%}r (“EM131”) inhibited SarsCoV-2 and had virucidal activity as shown in FIGS. 12, 13, 14 and 15, respectively.

### Example 4

### Inhibition of HSV-2

A. Toxicity assay on Vero Cells: Cytotoxicity of the dendritic polyglycerol is tested on mammalian cells. Vero cells are plated 24 h before the experiment in 96-well plates in order to have a confluent layer. Cells are then incubated with different concentrations of the compound being tested at 37° C. for 24 h in DMEM w2% FBS. The solution is then removed and the cells washed with DMEM w2% FBS. 100 ul of DMEM w2% FBS is added in each well with 20 ul of MTS (CellTiter 96® AQueous One Solution Cell Proliferation Assay). After 4 hours of incubation at 37° C., the absorbance of each well is measured through a plate-reader (k=490 nm). A percentage of cytotoxicity was then calculated comparing the absorbance with a reference, in which cells were incubated with just DMEM w2% FBS.

B. Inhibition assay against HSV-2: The antiviral effect of dendritic polyglycerol against HSV-2 is tested by plaque reduction assays on Vero cells. Vero cells are plated 24 h before the experiment in 24-well plates at a density of 10⁵ cells. A fixed amount of virus (MOI=0.0005) is pre-incubated for 1 hour with serial dilutions of the compound of interest. The solution is then transferred onto the cells and incubated for 1 hour. Afterwards, the solution is removed and the cells incubated for 24 h in DMEM w2% FBS with 0.45 w % Methyl-Cellulose. The cells are then stained with crystal violet and the plaques counted. The inhibition at each concentration is then compared to an untreated control and percentage of inhibition calculated.

C. Virucidal assay against HSV-2: The virucidal activity of the dendritic polyglycerol against HSV-2 is tested by virucidal assay. Vero cells are plated 24 h before the experiment in 96-well plates in order to have a confluent layer. An effective amount of dendritic polyglycerol (100-300-500 μg/ml) is incubated with a fixed amount of viruses (105-10⁶ pfu/ml) for 1 hour at 37° C. in DMEM-2% FBS. A serial dilution of this solution is added in each well and incubated for 1 hour at 37° C. Afterwards, the solution is removed and the cells incubated for 24 h in DMEM w2% FBS with 0.45 w % Methyl-Cellulose. The cells are then stained with crystal violet and the plaques counted. The viral titer is evaluated and compared against a reference with no compound.

D. Data Analysis: The EC₅₀ values for inhibition curves (dose-response assay) are calculated using GraphPad Prism 8.0 using a 4-parameter.

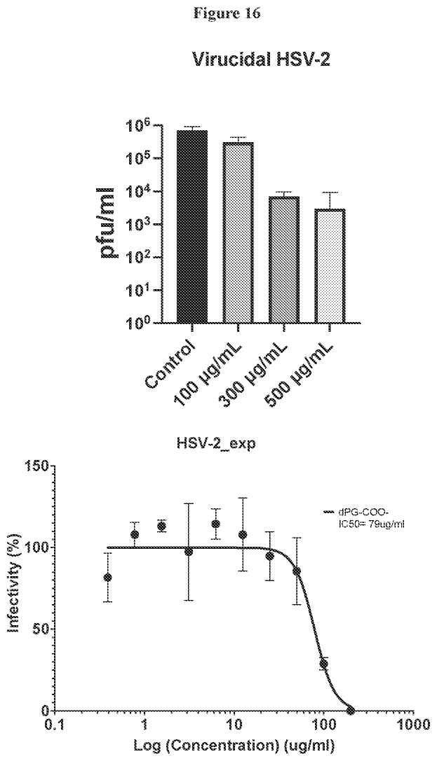

E. Results: The compound of the disclosure, dPG_{10 kDa}-C₃—S—C₁₀—COO⁻Na⁺_{100%}, when tested, for example as described above, was found to have an IC₅₀ of about 79 μg/ml and to have virucidal activity against HSV-2, as shown in FIG. 16.

### Example 5

### In Vivo Testing in the Syrian Hamster Model

Hamsters 6 wk to 10 wk old are anesthetized with ketamine/xylazine/atropine and inoculated intranasally with 50 μL containing 2×106 TCID50. Test compound is administered daily for 4 days intranasally (100 μl in PBS). Two different doses are tested: 1.5 mg/kg/day and 4.5 mg/kg/day. A placebo is used (100 μl of PBS) as a negative control. MK-4482 [an orally administered bioavailable prodrug (5′-isobutyric ester form) of the cytidine nucleoside analog EIDD-1931 (β-D-N4-hydroxycytidine; NHC)] is used as a positive control. MK-4482 is administered twice a day via oral gavage (187 μl) in formulation (MK-4482 at 100 mg/ml in 85:10:2.5 water:PEG400:Chremophor) as reported in Rosenke et al. Hamsters are monitored daily for appearance, behavior, and weight. At day 14 hamsters are euthanized and tissues [lungs, small intestine (ileum)] are collected. Viral RNA and infectious virus are quantified by RT-qPCR and end-point virus titration, respectively.

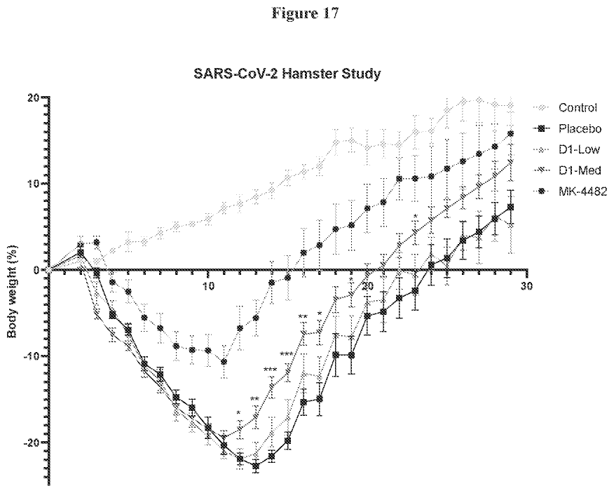

When tested as described above, compounds of Formula I are effective in treating hamsters infected with SARS-CoV-2, including inhibition of weight loss. FIG. 17 shows the results of testing dPG_{10 kDa}-C₃—S—C₁₀—COO⁻Na⁺_{100%}, where the control is indicated by a grey circle, placebo is indicated by a solid square, D1-Low represents 1.5 mg/kg/day of dPG_{10 kDa}-C₃—S—C₁₀—COO⁻Na⁺_{100%} and is indicated by an upward-pointing triangle, D1-Med represents 4.5 mg/kg/day of dPG_{10 kDa}-C₃—S—C₁₀—COO⁻Na⁺_{100%} and is indicated by a downward-pointing triangle, and MK-4482 is indicated by a solid black circle.

### Example 6

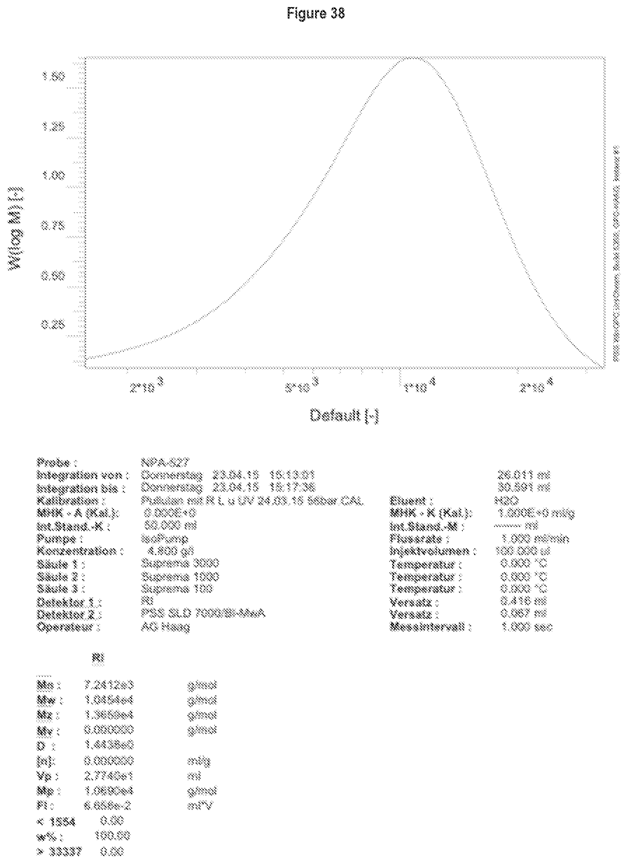

A. Dendritic polyglycerols synthesis (C1) A series of compounds have been synthesized through the chemical modification or functionalization of dendritic polyglycerols (dPGs). The dPG core was synthesized through slow monomer addition for ring opening multi-branching polymerization of glycidol (FIG. 1). In the first step, trimethylolpropane (TMP) (25 mmol) was deprotonated by potassium methoxide solution (10 mmol KOH in 10 mL methanol). The resulting methanol was evaporated at 60° C. under vacuum (3 mbar). The synthesis-reactor was then heated to 100° C. and glycidol was added slowly over a period of 24 h. The resulting dPG, having an average molecular weight of 10 kDa (“dPG_{10 kDa}” or “C1”), was used for functionalization. Molecular weight was determined by GPC as shown in FIG. 38. By varying the amount of glycidol and reducing the period of addition, the corresponding dPG, having a molecular weight of 5 kDa (“dPG_{5 kDa}”) was similarly obtained.

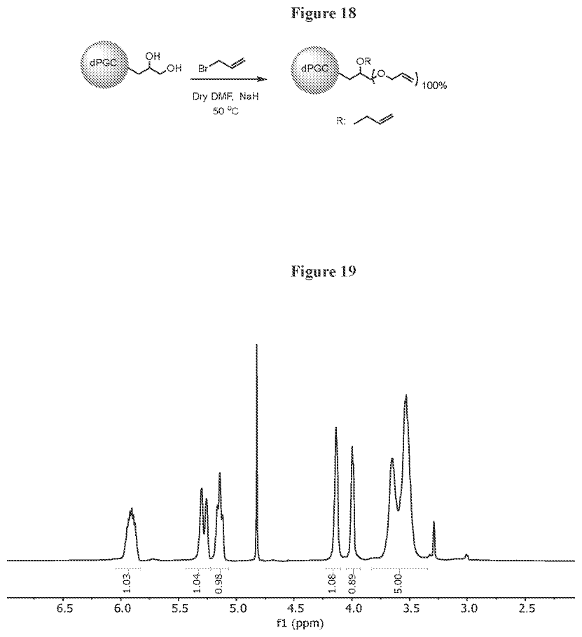

B. Synthesis of allyl-functionalized dPG (dPG_{10 kDa}-allyl) In order to prepare a soft material platform for the further thiol-ene click reaction, the free hydroxyl groups of dPG_{10 kDa} were converted to allyl groups through reaction with allyl bromide. The reaction was performed overnight in dry condition in presence of NaH as base for deprotonation of hydroxyl groups (FIG. 18). The DMF was removed in vacuum and the functionalized polymer (dPG_{10 kDa}-allyl) was purified by dialysis in MeOH for 2 days. The degree of functionalization was confirmed to be 100% by ¹H NMR of the pure product correlating the allyl protons at 6.0-5.1 ppm with the polyglycerol backbone protons (3.7-3.4) (FIG. 19). By limiting the equivalents of allyl bromide and NaH, a product, corresponding to the product in FIG. 2 where the degree of functionalization for the allyl group was 2%, was obtained.

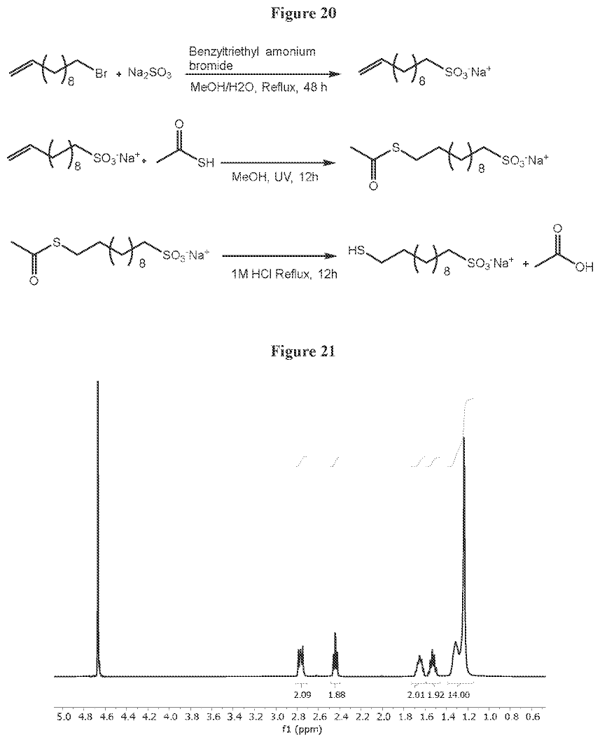

Synthesis of 11-mercapto-1-undecanesulfonate (MUS) In preparation to functionalize the surface of dPG_{10 kDa}-allyl, 11-mercapto-1-undecanesulfonate (MUS) was synthesized following a reported procedure (see, FIG. 20).

Sodium undec-10-enesulfonate: 11-bromo-1-undecene (25 mL, 111.975 mmol), sodium sulfite (Na₂SO₃) (28.75 g, 227.92 mmol) and benzyltriethyl-ammonium bromide (10 mg) were added to a mixture of 200 mL methanol and 450 mL DI-water in a 1 L round bottom flask. The mixture was refluxed at 102° C. for 48 h, extracted with diethyl ether 5 times (5×400 ml), and the aqueous phase was evaporated in a rotary evaporator. The resulting white powder was dried under high vacuum, suspended in pure ethanol and filtered. The solution was evaporated, and the process was repeated twice to decrease the amount of inorganic salts. About 33 g of sodium undec-10-enesulfonate was collected as a white, methanol soluble powder.

Sodium 11-acetylthio-undecanesulfonate: Sodium undec-10-enesulfonate (33 g, 147.807 mmol) was dissolved in 500 ml of methanol. (The resulting solution should be clear in order to have high yield, and any precipitate should be removed by filtration.) A 2.6 times excess of thioacetic acid (27.324 mL, 384.3 mmol) was added to the solution and it was stirred in front of a UV lamp overnight (12 h). The solution was evaporated in a rotary evaporator until the solid residue turned orange-red. The solid was washed with diethyl ether, until no colored material could be removed. The solid was dried under high vacuum, and then dissolved in methanol producing a yellow solution. About 3 g of carbon black was added to the solution, vigorously mixed, and the mixture was filtered through celite in a fluted filter paper. The filtered solution was clear, the solvent completely evaporated and about 35 g of sodium 11-acetylthio-undecanesulfonate was collected as a white solid.

11-Mercapto-1-undecanesulfonate (MUS): Sodium 11-acetylthio-undecanesulfonate (35 g, 120.7 mmol) was refluxed in 400 mL of 1M HCl for 12 h, after which 200 mL of 1 M NaOH was added to the resulting solution, and an additional 400 mL of DI-water was added to create a 1 L volume. The resulting clear solution was kept at 4° C. and crystallized overnight to yield a viscous white product that was centrifuged down in 50 mL falcon tubes, and dried under high vacuum. 12 g of methanol soluble MUS was collected from this purification step. (More material can be extracted from the supernatant of the centrifugation step, by reducing volume and keeping it at 4° C.) The successful synthesis of MUS was proved by ¹HNMR and ESI-MS analysis (ESI-MS m/z 313.08 (M+Na) and m/z 601.17 (2M+Na) for dimer which is formed due to disulfide bond formation).

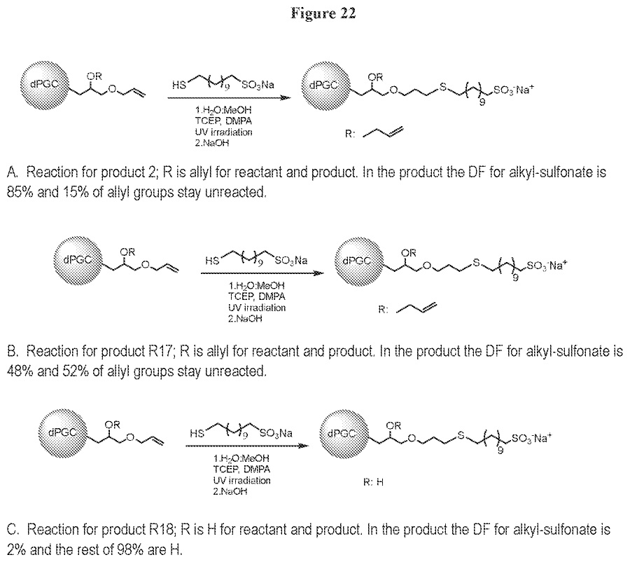

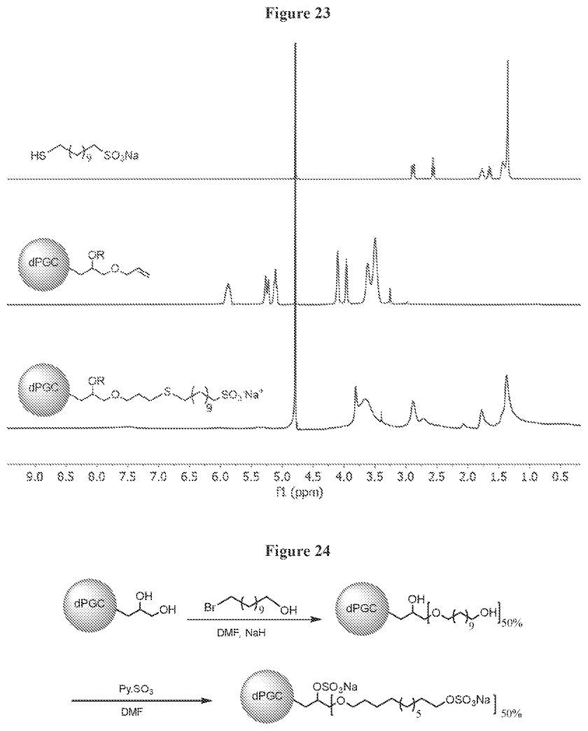

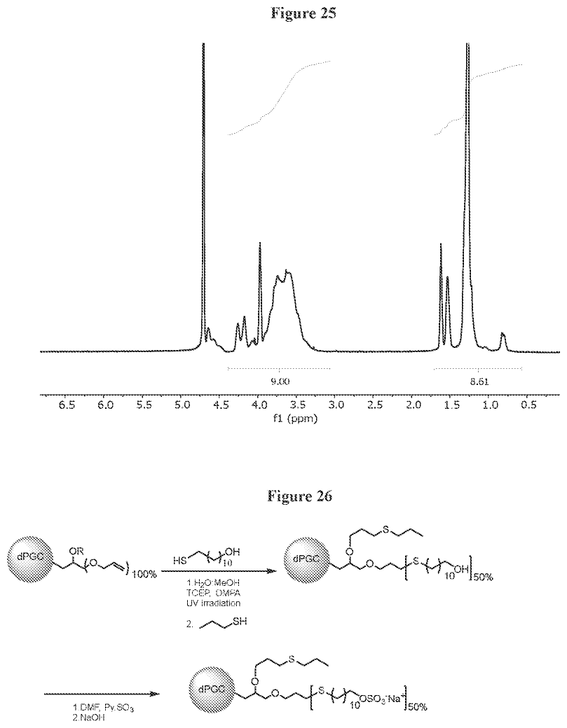

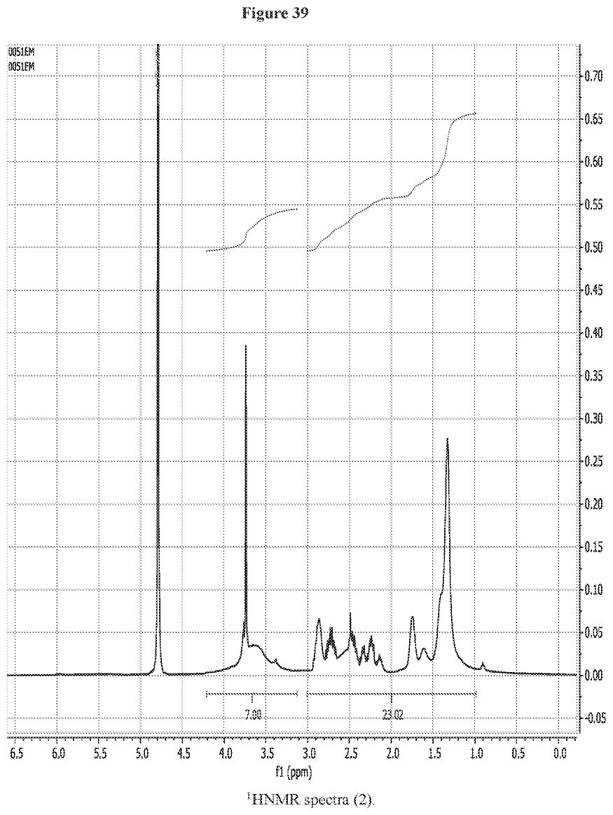

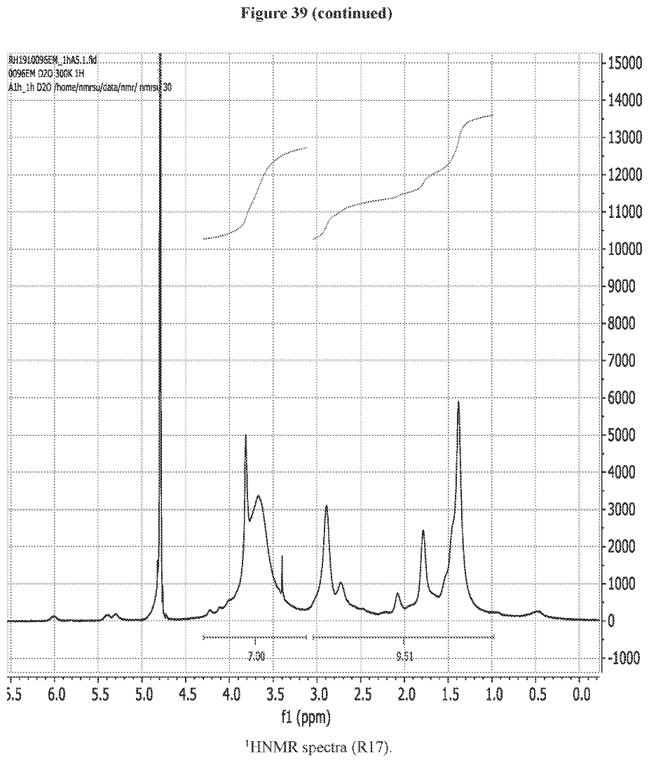

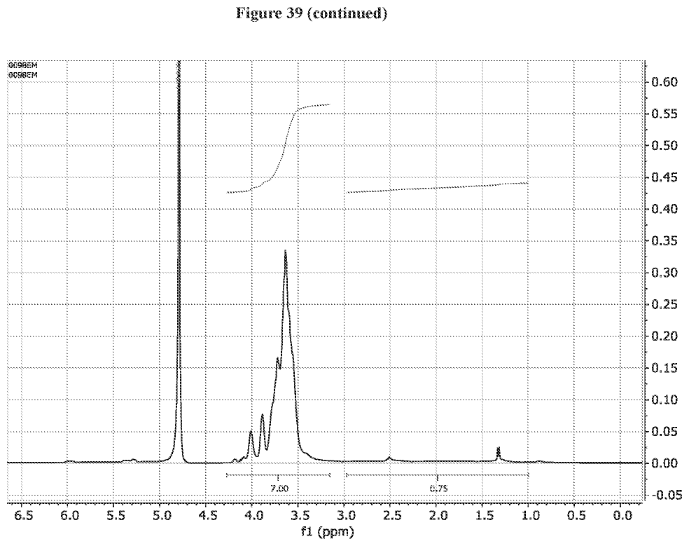

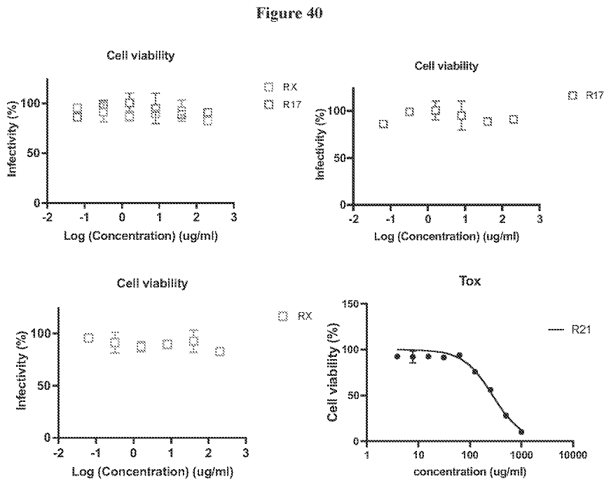

D. Synthesis of MUS functionalized dPG_{10 kD}a (dPG_{10 kDa}MUS) (Effect of different functionalization with C₁₁-sulfonate) (2(85%), R17(48%), R18(2%)) As illustrated in FIG. 22, the MUS moieties (obtained, for example, in Example 1C) were conjugated to the dPG_{10 kDa}allyl core (obtained, for example, in Example 1B) through the thiol-ene click reaction to obtain different degrees of functionalization. For this aim dPG_{10 kDa}allyl, MUS and 2,2-dimethoxy-2-phenylacetophenone (DMPA) as radical initiator were dissolved in a water:methanol mixture. A catalytic amount of tris(2-carboxyethyl)phosphine hydrochloride (TCEP-HCl) was added to reduce the disulfide bonds and avoid oxidation of the thiol intermediate. The solution was degassed by flushing argon through the reaction mixture for 10 minutes. The reaction mixture was stirred and irradiated with UV light using a high-pressure UV lamp at room temperature for 6 hours. The solution was then dialyzed (MWCO 2 kDa) against water:methanol for 2 days. The methanol was evaporated under reduced pressure and the samples were lyophilized to obtain the resulting products, dPG_{10 kDa}-MUS₈₅% (2), dPG_{10 kDa}-MUS_{48%} (R17) and dPG_{10 kDa}-MUS_{2%} (R18), as white solids. These three compounds with different degree of functionalization (85%, 48% and 2%) were synthesized, in the case of (2) and (R17) by varying the amount of MUS employed in the reaction, and in the case of (R18) by starting with a dPGC where degree of functionalization for the allyl group corresponding to R was 2% and the rest remained hydrogen (as discussed in the second paragraph of Example 1B). The degree of functionalization was measured by ¹HNMR (see, FIG. 39) and elemental analysis.

E. Synthesis of sulfate functionalized dPG (dPG-C₁₁-sulfate) (RX, R21) In order to investigate the effect of sulfate functional groups on the virucidal activity, dPG was functionalized with undecanesulfate, as illustrated in FIG. 24, starting with dPG_{5 kDa} and dPG_{10 kDa}. Each starting dPG (400 mg, 5.4 mmol of OH to be functionalized) was first reacted with 11-bromo-1-undecanol (2 g, 8.1 mmol, 1.5 eq.) in the presence of NaH (259.17 mg, 10.8 mmol, 2 eq.) as the base for deprotonation of the dPGC's hydroxyl groups to obtain the corresponding dPG-C₁₁—OH with 50% of degree of functionalization. Each reaction mixture was allowed to stir for 24 hours at 40° C. and then was quenched by adding methanol and purified by dialysis against methanol. In the next step, both types of hydroxyl groups (the 50% that converted to hydroxyalkyl and the 50% that remained OH) were sulfated through the reaction with pyridine sulfur trioxide complex in dry DMF at 60° C. overnight. The reactions were quenched with water, and the pH adjusted to 8 by addition of NaOH solution. Solvent was evaporated under reduced pressure, and the products, dPG_{5 kDa}-C₁₁-sulfate_{41%}/sulfonate_{59%} (overall weight 16 kDa) (R21) and dPG_{10 kDa}-C₁₁-sulfate_{50%}/sulfonate_{50%} (overall weight 30 kDa) (RX) were dissolved in brine. Dialysis was performed with a NaCl solution, using an ever-decreasing NaCl concentration, until the medium was changed with distilled water. Degree of functionalization was roughly 50% determined by ¹HNMR of the pure products correlating the alkyl chain protons at 1.7-0.5 ppm with the polyglycerol backbone protons (4.4-3.1). Size was determined by DLS as 140 nm for RX and 100 nm for R21.

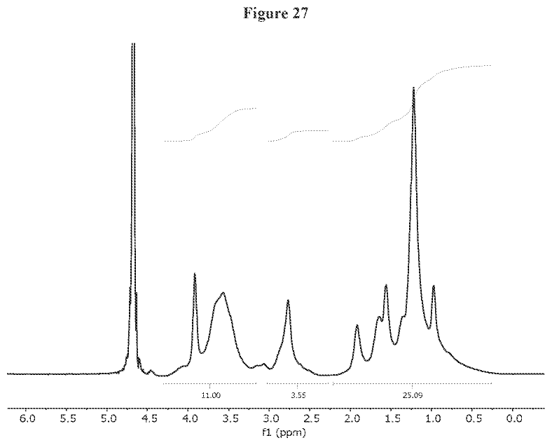

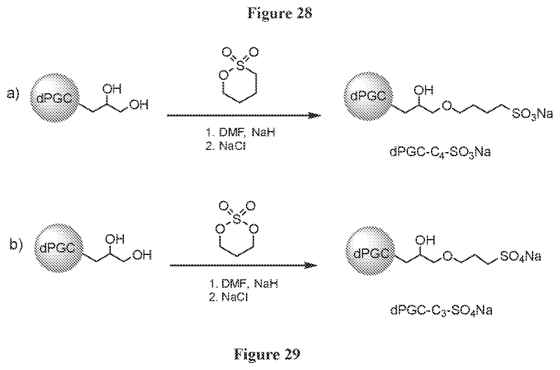

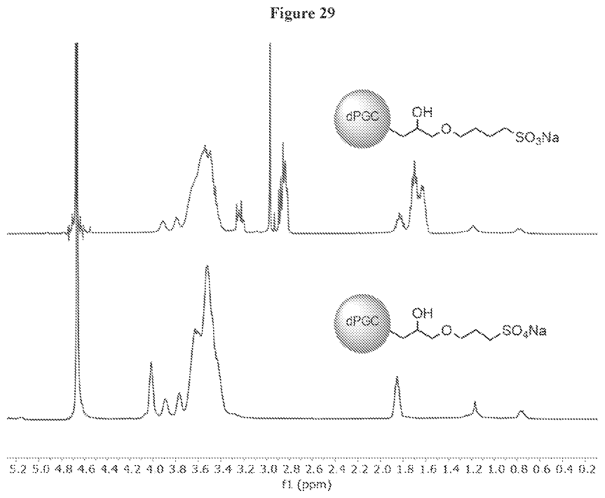

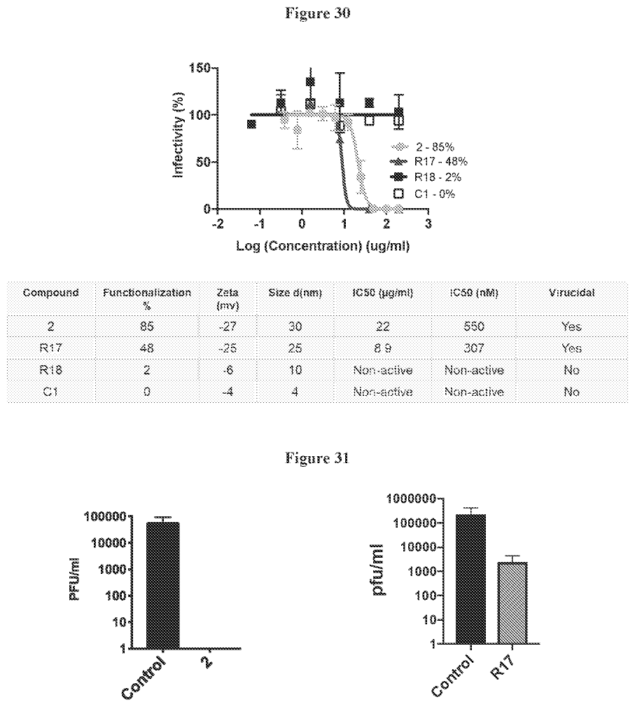

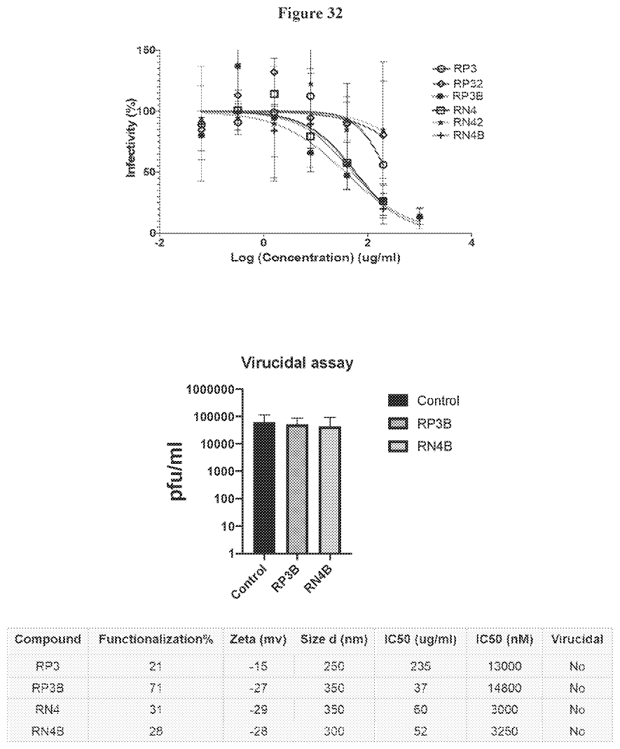

F. Synthesis of sulfate functionalized dPG with Sulfur Bridge (dPG-S—C11-sulfate) (comparison of sulfate and sulfonate) (R19B) In order to have a compound similar to R17 and compare the sulfate and sulfonate groups in term of virucidal activity, compound R19B was synthesized with similar degree of functionalization, as illustrated in FIG. 26 (where R in the starting reactant is allyl). 11-Mercapto-1-undecanol (MUD) moieties were conjugated to a dPG-allyl core through the thiol-ene click reaction aiming for 50% functionalization. The reaction started by dissolving dPG-allyl (50 mg, 0.67 mmol of allyl group), MUD (80 mg, 0.39 mmol) and catalytic amount of 2,2-dimethoxy-2-phenylacetophenone (DMPA) as a radical initiator in a water:methanol mixture. A catalytic amount of tris(2-carboxyethyl)phosphine hydrochloride (TCEP-HCl) was added to avoid oxidation of the thiol intermediate. The solution was degassed by flushing argon through the reaction mixture for 10 minutes. The reaction mixture was stirred and irradiated with UV light using a high-pressure UV lamp at room temperature for 4 hours and then 1-propanthiol was added to the mixture following by UV irradiation to quench the remaining allyl group (“R” in FIG. 26). The solution was then dialyzed (MWCO 2 kDa) against water:methanol for 2 days. The solvent was evaporated under reduced pressure to obtain a highly viscose white compound. In the next step, the hydroxyl group was sulfated through the reaction with pyridine sulfur trioxide complex in dry DMF at 60° C. overnight. The reaction was quenched with water, and the pH was adjusted to 8 by addition of NaOH solution. The solvent was evaporated under reduced pressure, and the product was dissolved in brine. Dialysis was performed with a NaCl solution, using an ever-decreasing NaCl concentration, until the medium was changed with distilled water. The final product, dPG_{10 kDa}-C₃SC₁₁-sulfates_{50%}/C₃SC_{3 50%} (R19B) was obtained as white solid compound after lyophilization (¹HNMR spectrum in FIG. 27).

G. Synthesis of sulfate and sulfonate functionalized dPG with short alkyl chains (C₃, C₄) (RP3, RN4) FIG. 28 illustrates a one-step ring opening functionalization. The starting material, dPG_{10 kDa} (100 mg, 1.35 mmol OH to be functionalized) was dried at 60° C. overnight under high vacuum. The dried dPG was dissolved in dry DMF (5 mL). To the stirred solution of dPG in dry DMF at room temperature, NaH (65 mg, 2.7 mmol, 2 eq.) was added. The reaction mixture was allowed to stir for 1 hour at room temperature. At this point, the syntheses diverge for production of the two products, proceeding in separate reaction vessels, to which 1,4-butane sultone (276 μL, 2.7 mmol, 2 eq.) (for RP3) or 1,3-propanediol cyclic sulfate (373.3 mg, 2.7 mmol, 2 eq.) (for RN4) were added and the reactions were stirred overnight at room temperature. Each mixture was then dialysed against brine using an ever-decreasing NaCl concentration for 2 days, until the medium was changed with distilled water and dialysis continued for 2 more days. The solvent was decreased under reduced pressure and the respective products, dPG_{10 kDa}-C₃-sulfate_{21%} (RP3) and dPG_{10 kDa}-C₄-sulfonate_{31%} (RN4) were obtained as crystalline powders after lyophilisation.

### Example 7

### Characterization

A. Gel permeation chromatography (GPC) GPC measurements were performed using an Agilent 1100 solvent delivery system with a manual injector, isopump, and Agilent 1100 differential refractometer. A Brookhaven BIMwA7-angle light scattering detector was coupled to size exclusion chromatography (SEC) to measure the molecular weight of each fraction of the polymer that was eluted from the SEC columns. For the separation of the polymer samples, three 30 cm columns were used (10 μm PSS Suprema columns with pore sizes of 100 Å, 1000 Å, and 3000 Å). Water was used as the mobile phase; the flow rate was set at 1.0 mL min⁻¹. All columns were held at room temperature. For each measurement, 100 μL of sample with a concentration of 5 mg mL⁻¹ was injected. For acquisition of the data from seven scattering angles (detectors), a differential refractometer WinGPC Unity from PSS was used. Molecular weight distributions were determined by comparison with pullulan standards (10 different sizes from 342 to 710 000 g mol⁻¹). Water was used as a solvent with 0.1 M NaNO₃.

B. Dynamic light scattering (DLS) The size of the dendritic polyglycerols was measured in aqueous solution using a Zetasizer Nano ZS analyzer with an integrated 4 mW He—Ne laser at a wavelength of 633 nm with a backscattering detector angle of 1730 (Malvern Instruments Ltd, UK) at 25° C. For DLS experiments, an aqueous solution of dPG with different concentrations was prepared in Milli-Q water and vigorously stirred for 18 hours at room temperature (25° C.). Solutions were filtered via 0.45 μm polytetrafluoroethylene (PTFE) filters and used for dynamic light scattering measurements. Disposable UV-transparent cuvettes (Sarstedt AG & Co, Germany) were used for all the experiments.

C. Nuclear magnetic resonance (NMR) NMR spectra were recorded on a Jeol ECX 400 or a Jeol Eclipse 700 MHz spectrometer. Proton NMR spectra were recorded at 295 K in ppm and were referenced to the indicated solvents.

### Example 8

Other Compounds of Formula I

A. By following the procedures of Example 1A and adjusting the equivalents of glycidol and reaction time accordingly, there are obtained dPG_{5 kDa}, dPG_{25 kDa}, dPG_{50 kDa}, dPG_{75 kDa}, and dPG_{100 kDa}.

B. By following the procedures of Example 1B and substituting allyl bromide with:
a) 4-bromobut-1-ene,
b) 5-bromopent-1-ene,
c) 7-bromo-5-methylhept-1-ene, and
d) 10-iodo-5-isopropyl-6-methyldec-1-ene;
   there are obtained the following compounds, respectively:
a) dPG_{10 kDa}-but-1-ene₁₀₀%,
b) dPG_{10 kDa}-pent-1-ene₁₀₀%,
c) dPG_{10 kDa}-5-methylhept-1-ene_{100%}, and
d) dPG_{10 kDa}-5-isopropyldec-1-ene₁₀₀%.

C. By following the procedures of Example 1C and substituting 11-bromo-1-undecene with:
a) 8-bromooct-1-ene,
b) 11-bromo-5,4-dimethylundec-1-ene,
c) 14-bromotetradec-1-ene,
d) 8-ethyl-16-iodo-5-methylhexadec-1-ene, and
e) 26-bromohexacos-1-ene;
   there are obtained the following compounds, respectively:
a) sodium 8-mercaptooctane-1-sulfonate,
b) sodium 11-mercapto7,8-dimethylundecane-1-sulfonate,
c) sodium 14 mercaptotetradecane-1-sulfonate,
d) sodium 9-ethyl-16-mercapto-12-methylhexadecand-1-sulfonate, and
e) sodium 26-mercaptohexacosand-1-sulfonate.

D. By following the procedures of Example 1D and substituting sodium 11-mercapto-1-undecane sulfonate with:
a) sodium 8-mercaptooctane-1-sulfonate,
b) sodium 11-mercapto7,8-dimethylundecane-1-sulfonate,
c) sodium 14 mercaptotetradecane-1-sulfonate,
d) sodium 9-ethyl-16-mercapto-12-methylhexadecane-1-sulfonate, and
e) sodium 26-mercaptohexacosand-1-sulfonate;
   there are obtained the following compounds, respectively:
a) dPG_{10 kDa}-C₃—S—C₈SO₃⁻Na⁺_{85%},
b) dPG_{10 kDa}-C₃—S—C₁₁-7,8-di-Me-SO₃⁻Na⁺_{85%},
c) dPG_{10 kDa}-C₃—S—C₁₁SO₃—Na⁺_{48%},
d) dPG_{10 kDa}-C₃—S—C₁₆-9-Et-12-Me-SO₃⁻Na⁺_{48%}/allyl_{52%}, and
e) dPG_{10 kDa}-C₃—S—C₂₆SO₃⁻Na⁺_{85%}/allyl_{15%}.

E. By following the procedures of Example 1E and substituting 11-bromo-1-undecanol with:
a) 5-bromopentan-1-ol,
b) 7-bromoheptan-1-ol,
c) 7-iodo-2-methylheptan-1-ol,
d) 8-bromooctan-1-ol,
e) 9-bromononan-1-ol,
f) 10-bromodecan-1-ol,
g) 11-bromo-4,5-dimethylundecan-1-ol,
h) 12-chlorododecan-1-ol,
i) 13-bromotridecan-1-ol,
j) 14-bromotetradecan-1-ol,
k) 15-bromopentadecan-1-ol,
1) 18-iodooctadecan-1-ol, and
m) 31-bromohentriacontan-1-ol;
   there are obtained the following compounds, respectively:
a) dPG_{10 kDa}-C₅—SO₄⁻Na⁺_{50%}/SO₃⁻Na⁺_{50%},
b) dPG_{10 kDa}-C₇—SO₄⁻Na⁺_{50%}/SO₃⁻Na⁺_{50%},
c) dPG_{10 kDa}-C₇-2-Me-SO₄⁻Na⁺_{50%}/SO₃⁻Na⁺_{50%},
d) dPG_{10 kDa}-C₈—SO₄⁻Na⁺_{50%}/SO₃⁻Na⁺_{50%},
e) dPG_{10 kDa}-C₉—SO₄⁻Na⁺_{50%}/SO₃⁻Na⁺_{50%},
f) dPG_{10 kDa}-C₁₀-SO₄⁻Na⁺_{50%}/SO₃⁻Na⁺_{50%},
g) dPG_{10 kDa}-C₁₁-4,5-di-Me-SO₄⁻Na⁺_{50%}/SO₃⁻Na⁺_{50%},
h) dPG_{10 kDa}-C₁₂—SO₄⁻Na⁺_{50%}/SO₃⁻Na⁺_{50%},
i) dPG_{10 kDa}-C₁₃—SO₄⁻Na⁺_{50%}/SO₃⁻Na⁺_{50%},
j) dPG_{10 kDa}-C₁₄—SO₄⁻Na⁺_{50%}/SO₃⁻Na⁺_{50%},
k) dPG_{10 kDa}-C₁₅—SO₄⁻Na⁺_{50%}/SO₃⁻Na⁺_{50%},
l) dPG_{10 kDa}-C₁₈—SO₄⁻Na⁺_{50%}/SO₃⁻Na⁺_{50%}, and
m) dPG_{10 kDa}-C₃₁—SO₄⁻Na⁺_{50%}/SO₃⁻Na⁺_{50%}.

F. By following the procedures of Example 1F and substituting 11-mercapto-1-undecanol with:
a) 8-mercaptooctan-1-ol,
b) 10-mercaptodecan-1-ol,
c) 1-hydroxy-11-mercaptoundecyl sulfate,
d) 12-mercaptododecan-1-ol, and
e) 13-mercaptotridecan-1-ol;
   there are obtained the following compounds, respectively:
a) dPG_{10 kD}-C₃SC₈—SO₄⁻Na⁺_{50%}/C₃SC_{3 50%},
b) dPG_{10 kDa}-C₃SC₁₀—SO₄⁻Na⁺_{50%}/C₃SC_{3 50%},
c) dPG_{10 kDa}-C₃SC₁₁-di-SO₄⁻Na⁺_{50%}/C₃SC_{3 50%},
d) dPG_{10 kDa}-C₃SC₁₂—SO₄⁻Na⁺_{50%}/C₃SC_{3 50%}, and
e) dPG_{10 kDa}-C₃SC₁₃—SO₄⁻Na⁺_{50%}/C₃SC_{3 50%}.

### Example 6

### Test Results

A. Sulfonated dendritic polyglycerol (2, R17, R18) Sulfonated dendritic polyglycerols with different degrees of functionalization were tested against HSV-2. As shown in FIG. 30, the bare dendritic polyglycerol (C1) did not show any inhibitory activity in the range tested, as well as the dendritic polyglycerol having a low functionalization (R18, 2%, namely 4 groups per dendritic polyglycerol). Functionalization of at least 50% (2) and (R17) gave inhibitory activity with an IC₅₀ in the order of hundreds of nanomolar (˜300-550 nM). Both the compounds having a DF of at least 50% show virucidal activity, as can be seen in FIG. 31, where a decrease of the viral titer of more than 2 orders of magnitude is shown for both the compounds.

B. Alkyl chain length—(RP3, RN4) Dendritic polyglycerols with shorter alkyl chain (C₃ and C₄), bearing either sulfonate (RP3) or sulfate (RN4) functionality show limited efficacy against HSV-2 as reported in FIG. 32. Indeed, neither of them show virucidal activity. This result confirms the importance of the length of the linker to obtain a strong interaction between the dPG and the virus.

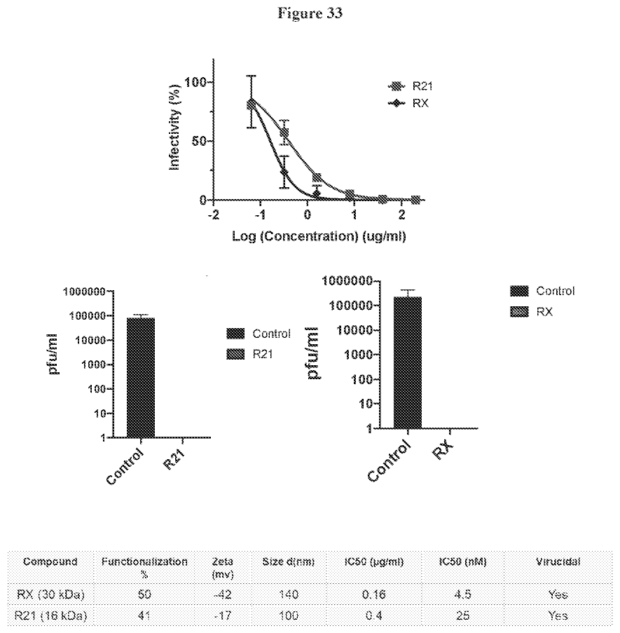

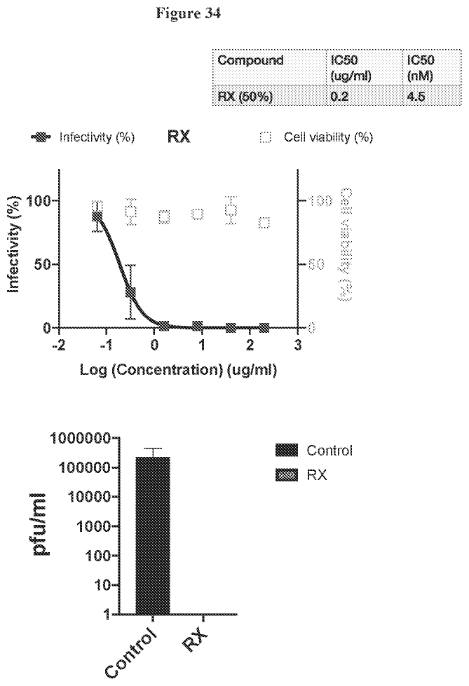

C. Size and weight (RX, R21) The dendritic polyglycerols RS and R21 were tested for inhibition of HSV-2 as described in Example 4A2 and for virucidal activity as described in Example 4A3. This evidences the impact of molecular weight (and size) on activity. The results are shown in FIG. 33. The results for RX are also shown in FIG. 34. Both of the compounds showed excellent virucidal inhibitory activity against HSV-2, having IC₅₀ in the nanomolar range. Both compounds are virucidal. RX, a slightly larger version, proved to be more effective than the smaller one (R21), having an IC50 of 4.5 nM.

D. Sulfate/Sulfonate (RX, R19B) The compound R19B was tested to evaluate the activity of sulfated dPGs. The results are reported in FIG. 35. The results show an extremely low IC50, but not as low as RX (as shown in the table). The difference in overall zeta-potential may correlate with the higher inhibition activity of RX as compared to R19B.

E. Comparison with CD and AuNPs Gold nanoparticles covered with a binary shell of MUS:OT and modified cyclodextrins bearing MUS have recently been reported as having broad-spectrum antiviral activity with no toxicity and virucidal activity, but these approaches have their own limitations. The main limitation of the gold nanoparticles is that the presence of the gold core raises concerns about bio-accumulation. On the other side, the gold nanoparticles have an antiviral activity in the nanomolar range. Conversely, modified cyclodextrins are based on an FDA-approved organic core that overcomes the bio-accumulation issues of the gold nanoparticles, but the modified cyclodextrin's activity is much lower (almost 3 order of magnitude), being in the micromolar range.

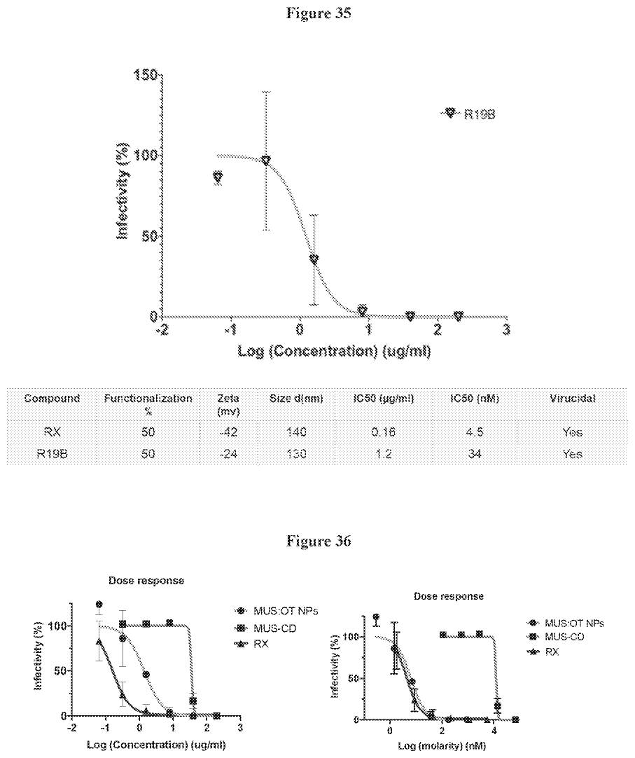

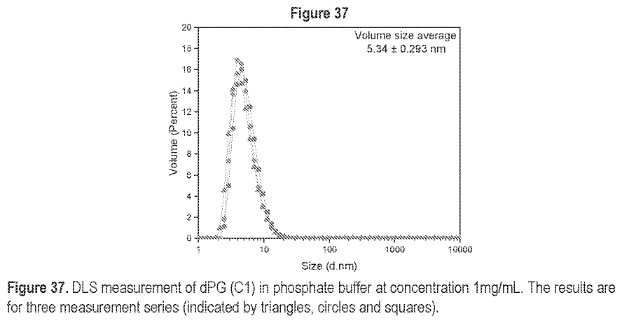

RX has been compared against these two other compounds. FIG. 36 shows the results of testing inhibition activity of the three compounds, e.g., as described in Example 4A2. The data, reported in Table 1, show that RX is superior to both compounds in terms of IC₅₀ (both in molarity and in mass concentration), meaning that RX has a stronger affinity. Thus, RX has an equivalent effect on HSV-2 as compared to AuNPs and MUS-CD, respectively, using 1/10 and 1/200 of the material.

TABLE 1

| | | Molecular | | |
|---|---|---|---|---|
| | Active | weight | IC50 | IC50 |
| Compound | groups | (Da) | (ug/ml) | (nM) |
| MUS:OT NPs | 58 | 220000 | 1.4 ± 0.8 | 6.5 ± 4 |
| MUS-CD | 7 | 2880.36 | 35 ± 0.05 | 12400 |
| RX | 65 | 36000 | 0.16 ± 0.05 | 4.5 ± 1 |

F. Summary Table 2 summarizes the characteristics and activities of the compounds made and tested as reported above.

TABLE 2

| | dPGC | | | | | | |
|---|---|---|---|---|---|---|---|
| | Weight | Size | | | | IC₅₀ | |
| Name | kDa | nm | R | DF % | R not in DF | nm | Virucidal |
| C1 | | 4 | H | 0 | H | N/A | N |
| 2 | 10 | 30 | —(CH₂)₃—S— | 85 | H or | 550 | Y |
| | | | (CH₂)₁₁—SO₃ | | —CH₂—CH═CH₂ | | |
| R17 | 10 | 25 | —(CH₂)₃—S— | 48 | H or | 307 | Y |
| | | | (CH₂)₁₁—SO₃ | | —CH₂—CH═CH₂ | | |
| R18 | 10 | 10 | —(CH₂)₃—S— | 2 | H or | N/A | N |
| | | | (CH₂)₁₁—SO₃ | | —CH₂—CH═CH₂ | | |
| R19B | 10 | 130 | —(CH₂)₃—S— | 50 | —(CH₂)₃—S— | 34 | Y |
| | | | (CH₂)₁₁—OSO₃ | | (CH₂)₂—CH₃ | | |
| R21 | 5 | 100 | —(CH₂)₁₁—OSO₃ | 41 | SO₃ | 25 | Y |
| RX | 10 | 140 | —(CH₂)₁₁—OSO₃ | 50 | SO₃ | 4.5 | Y |
| RN4 | 10 | 350 | —(CH₂)₃—OSO₃ | 31 | H | 3000 | N |
| RP3 | 10 | 250 | —(CH₂)₄—SO₃ | 21 | | 13000 | N |

### Example 7

### Lip Balm Formulation

TABLE 3

| | | Specific | Amount | % |
|---|---|---|---|---|
| Ingredient | Amount | gravity | per unit | (w/w) |
| dPG_{10 kDa}-C₃SC₁₀- | 0.03335 g | | 0.00667 g | 0.1 |
| COO⁻Na⁺_{100%} | | | | |
| or | | | | |
| dPG RX | | | | |
| 2-Deoxy-D-glucose | 0.667 g | | 0.0133 g | 0.2 |
| Silica gel, USP/ | 0.200 g | | 0.04 g | 0.5 |
| NF powder | | | | |
| Stevioside, | 0.333 g | | 0.0667 g | 0.9 |
| 90% powder | | | | |
| Polyethylene glycol | 17.89 g | | 3.578 g | 48.0 |
| 1450, NF Granules | | | | |
| Polyethylene glycol | 16.667 | 1.1 | 3.33 | 49.1 |
| 300, NF Liquid | mL | | mL | |
| Flavor, spearmint oil | 0.5 | 0.917 | 0.1 | 1.2 |
| | mL | | mL | |

As disclosed in US 2013/0150312, polyethylene 1450 and 300 are melted at 50° C. with stirring. The dPG, 2-deoxy-D-glucose, silica gel and stevioside are triturated together. The triturated powders are slowly sifted into the melted PEGs with stirring. The flavoring is added, followed by thorough mixing. The mixture is poured into applicator tubes and allowed to cool to room temperature.

### Example 8

### Aqueous Cream Formulation

TABLE 4

| Ingredient | Amount |
|---|---|
| dPG_{10 kDa}-C₃SC₁₀- | 1.0 g |
| COO⁻Na⁺_{100%} | |
| or | |
| dPG RX | |
| 2-Deoxy-D-glucose | 2.0 g |
| Cetostearyl alcohol | 67.5 g |
| Sodium lauryl sulphate | 7.5 g |
| White soft paraffin | 125.0 g |
| Liquid paraffin | 50.0 g |
| Propylene glycol | 400.0 g |
| Purified water | q.s. to 1000 g |

As disclosed in US 2013/0150312, a part of the dPG is dissolved in water with the 2-deoxy-D-glucose and propylene glycol at ambient temperature to produce an aqueous solution. The paraffins and emulsifiers (cetostearyl alcohol and sodium lauryl sulphate) are mixed together, heated to 60° C., and emulsified with the aqueous solution, also at 60° C. The remaining dPG is added, the mixture dispersed, allowed to cool, and filled into lacquered aluminum tubes.

### Example 9

### Nebulizer Formulation

A nebuliser, for example, as disclosed in U.S. Pat. No. 9,364,618 B2 or EP 3,517,117 A1 is provided comprising the following pharmaceutical composition in its fluid reservoir: 14.0 mg dPG_{10 kDa}-C₃SC₁₀—COO⁻Na⁺_{100%} (or dPG RX), 0.9% w/v NaCl dissolved in sterile deionised water. The nebuliser is used to deliver the composition by inhalation as an aerosol to the lower respiratory tract of a patient suffering from influenza.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto. All patents and publications cited above are hereby incorporated by reference.

We claim:

1. A compound, pharmaceutically acceptable salt or pharmaceutically acceptable ester of Formula I: 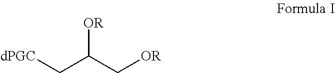
wherein:
dPGC is dendritic polyglycerol core in which OR represents the free OH groups within and at the peripheryu of the core, having an average molecular weight from 5 to 100 kDa as measured by GPC;
R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₅ to C₃₀ alkene)-COOH, —(CH₂)_{z}—O—(CH₂)_{y}—COOH, and —(CH₂)_{z}—S—(CH₂)_{y}—COOH, —SO₃⁻, —(CH₂)_{z}—S—(CH₂)_{y}—CH₃, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃⁻, —(C₅ to C₃₀ alkenyl)-SO₃⁻, -(optionally substituted C₅ to C₃₀ alkyl)-O—SO₃⁻, —(C₅ to C₃₀ alkenyl)-OSO₃⁻, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃;
y is an integer from about 4 to about 30;
z is an integer from 1 or 2 to about 20;
y+z is an integer from about 5 or 6 to about 30; and
having a DF of at least about 30%.

2. The compound, pharmaceutically acceptable salt or pharmaceutically acceptable ester of claim 1, wherein:
R can be the same or different and is selected from the group comprising: —H, —COOH, optionally substituted C₅ to C₃₀ alkyl, C₃ to C₃₀ alkene, -(optionally substituted C₅ to C₃₀ alkyl)-COOH, —(C₈ to C₃₀ alkene)-COOH, —(CH₂)_{z}—O—(CH₂)_{y}—COOH,
and —(CH₂)_{z}—S—(CH₂)_{y}—COOH;
y is an integer from about 4 to about 30;
z is an integer from 1 to about 20;
y+z is an integer from about 5 to about 30; and
having a DF of at least about 30% as measured by ¹HNMR, where R contributing to the DF has a —COOH.

3. The compound, pharmaceutically acceptable salt or pharmaceutically acceptable ester of claim 1, wherein:
R can be the same or different and is selected from the group comprising: —H, —SO₃⁻, —(CH₂)_{z}—S—(CH₂)_{y}—CH₃, -optionally substituted C₅ to C₃₀ alkyl, —C₅ to C₃₀ alkenyl, C₅ to C₃₀ ω-hydroxyalkyl, C₃ to C₃₀ ω-hydroxyalkenyl, C₅ to C₃₀ ω-hydroxyalkylthioalkyl, C₅ to C₃₀ ω-hydroxyalkoxyalkyl, C₅ to C₃₀ ω-haloalkyl, and C₅ to C₃₀ ω-haloalkoxyalkyl, -(optionally substituted C₅ to C₃₀ alkyl)-SO₃⁻, —(C₅ to C₃₀ alkenyl)-SO₃⁻, -(optionally substituted C₅ to C₃₀ alkyl)-OSO₃, —(C₅ to C₃₀ alkenyl)-OSO₃, —(CH₂)_{z}—O—(CH₂)_{y}—SO₃⁻, —(CH₂)_{z}—O—(CH₂)_{y}—O—SO₃⁻, —(CH₂)_{z}—S—(CH₂)_{y}—SO₃⁻, and —(CH₂)_{z}—S—(CH₂)_{y}—OSO₃;
y is an integer from about 4 to about 30;
z is an integer from about 2 to about 20;
y+z is an integer from about 6 to about 30; and
having a DF of at least about 30% as measured by 1 HNMR, where R contributing to the DF has an —SO₃⁻ or an —O—SO₃⁻.

4. The compound, pharmaceutically acceptable salt or pharmaceutically acceptable ester of claim 2, wherein R contributing to DF is selected from the group comprising: -(optionally substituted C₈ to C₁₅ alkyl)-COOH, —(CH₂)_{z}—O—(CH₂)_{y}—COOH, and —(CH₂)_{z}—S—(CH₂)_{y}—COOH.

5. The compound, pharmaceutically acceptable salt or pharmaceutically acceptable ester of claim 1, wherein y is from about 8 to 13, z is from about 2 to 5, and y+z is from about 10 to 16.

6. The compound, pharmaceutically acceptable salt or pharmaceutically acceptable ester of claim 5, wherein R contributing to DF is —(CH₂)_{z}—S—(CH₂)_{y}—COOH.

7. The compound, pharmaceutically acceptable salt or pharmaceutically acceptable ester of claim 1, wherein dPGC has an average molecular weight of about 5 kDa to 25 kDa.

8. The compound, pharmaceutically acceptable salt or pharmaceutically acceptable ester of claim 1, having a DF of at least about 50%.

9. A compound, pharmaceutically acceptable salt or pharmaceutically acceptable ester of claim 3, wherein R contributing to DF is selected from the group comprising: —(C₈ to C₁₅ alkyl)-SO₃H, —(C₈ to C₁₅ alkyl)-OSO₃H, —(CH₂)_{z}—S—(CH₂)_{y}—SO₃H, and —(CH₂)_{z}—S—(CH₂)_{y}—OSO₃H.

10. A compound, pharmaceutically acceptable salt or pharmaceutically acceptable ester of claim 3, wherein:
R contributing to DF is —(CH₂)₃—S—(CH₂)₁₁—SO₃⁻, —(CH₂)₁₁—OSO₃ or —(CH₂)₁₁—SO₃;
R not contributing to DF is H, SO₃, or —CH₂—CH═CH₂; and
DF is at least about 40%.

11. The compound, pharmaceutically acceptable salt or pharmaceutically acceptable ester of claim 1 for use in treating a viral infection or a disease associated with a virus.

12. The compound, pharmaceutically acceptable salt or pharmaceutically acceptable ester of claim 11 wherein the virus is SARS-CoV-2 or HSV-2.

13. The compounds, pharmaceutically acceptable salts or pharmaceutically acceptable esters for use of claim 11, wherein the virus is selected from the group comprising HIV-1, HSV, HCMV, HPV, Respiratory syncytial virus (RSV), influenza virus, and filoviruses.

14. A virucidal composition comprising an effective amount of a compound, pharmaceutically acceptable salt or pharmaceutically acceptable ester of claim 1 and a suitable carrier.

15. A method of disinfection and/or sterilization of non-living surfaces using a compound, pharmaceutically acceptable salt or pharmaceutically acceptable ester of claim 1.

16. A device comprising a surface coated with a compound, pharmaceutically acceptable salt or pharmaceutically acceptable ester of claim 1.
